(19) United States
(12) Patent Application Publication
(10) Pub. No.: US 2007/0293674 A1
(43) Pub. Date: Dec. 20, 2007

(54) Novel Saperconazole Crystalline Forms and Related Processes, Pharmaceutical Compositions and Methods

(75) Inventors: Lisa Scoppettuolo, Allston, MA (US); Matthew Peterson, Hopkinton, MA (US); Orn Almarsson, Shrewsbury, MA (US); Julius Remenar, Framingham, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: TRANSFORM PHARMACEUTICALS, INC., Lexington, MA (US)

(21) Appl. No.: 11/568,131

(22) PCT Filed: Apr. 21, 2005
(86) PCT No.: PCT/US05/13418

§ 371 Date: Aug. 21, 2007

Related U.S. Application Data
(60) Provisional application No. 60/564,357, filed on Apr. 22, 2004.
(60) Provisional application No. 60/569,036, filed on May 7, 2004.

(51) Int. Cl. C07D 403/14

(52) U.S. Cl. 544/366

(57) ABSTRACT

The invention provides novel soluble conazole crystalline forms (e.g., cisitraconazole, saperconazole) that include salts, co-crystals and polymorphs useful as pharmaceuticals. The invention also provides pharmaceutical compositions comprising, and processes for making, these conazole crystalline forms. Methods of using such compositions for the treatment or prevention of systemic and local fungal, yeast, and dermatophyte infections are also provided.

### CROSS-REFERENCE TO RELATED APPLICATIONS

The present application claims the benefit of priority of U.S. Provisional Application Ser. No. 60/564,357, filed Apr. 22, 2004, and U.S. Provisional Application Ser. No. 60/569,036, filed May 7, 2004, which are hereby incorporated by reference herein in their entirety, including any figures, tables or drawings.

### FIELD OF INVENTION

The invention provides novel soluble saperconazole and cis-itraconazole crystalline forms that include salts, co-crystals and polymorphs useful as pharmaceuticals. The invention also provides pharmaceutical compositions comprising, and processes for making, these saperconazole and cis-intraconazole crystalline forms. Methods of using such compositions for the treatment or prevention of systemic and local fungal, yeast, and dermatophyte infections are also provided. In several embodiments, the invention provides novel soluble crystalline systems comprising (a) an organic salt comprising the reaction product of saperconazole and an organic or inorganic acid; or (b) a co-crystal comprising the reaction product of saperconazole and an organic or inorganic acid.

In several embodiments of the invention, the novel soluble crystalline forms of saperconazole and cis-itraconazole are characterized by a powder X-ray diffraction pattern expressed in terms of 2 theta angles.

### BACKGROUND OF THE INVENTION

Systemic fungal diseases (systemic mycoses) are typically chronic conditions that develop very slowly. These diseases are often induced by opportunistic causative fungi that are not normally pathogenic and commonly live in the patient's body or are commonly found in the environment. While systemic fungal diseases used to be relatively rare in temperate countries, there has been an increasing incidence of numerous life-threatening systemic fungal infections that now represent a major threat to susceptible patients. Susceptible patients include immunocompromised patients, particularly those already hospitalized, and patients compromised by HIV infection, ionizing irradiation, corticosteroids, immunosuppressives, invasive surgical techniques, prolonged exposure to antimicrobial agents, and the like, or by diseases or conditions such as cancer, leukemia, emphysema, bronchiectasis, diabetes mellitus, burns, and the like. The symptoms manifested by these fungal diseases are generally not intense, and may include chills, fever, weight loss, anorexia, malaise, and depression.

The most common systemic fungal infections in humans are blastomycosis, candidosis, aspergillosis, histoplasmosis, coccidioidomycosis, paracoccidioidomycosis, and cryptococcosis.

Fungal diseases are often confined to typical anatomic sites, and many involve a primary focus in the lung, with more characteristic manifestations of specific fungal infections appearing once the infection spreads from a primary site. For example, blastomycosis primarily involves the lungs, and occasionally spreads to the skin. Similarly, the primary form of coccidioidomycosis occurs as an acute, benign, self-limiting respiratory disease, which can then progress to a chronic, often-fatal infection of the skin, lymph glands, liver, and spleen. Other infectious diseases such as paracoccidioidomycosis and candidiasis present in different manners, and depending on the etiology, may exhibit several forms involving internal organs, the lymph nodes, skin, and mucous membranes. Diagnosis of specific fungal diseases can be made by isolation of the causative fungus from various specimens, such as sputum, urine, blood, or the bone marrow, or with certain fungus types, through evidence of tissue invasion.

Many patients suffering from severe systemic fungal infections are hardly, or not at all, able to receive medication via oral administration, as such patients are often in a coma or suffering from severe gastroparesis. As a result, the use of insoluble or sparingly soluble antifingals such as itraconazole free base, which are difficult to administer intravenously to treat such patients, is significantly impeded.

Local or superficial fungal infections are caused by dermatophytes or fungi that involve the outer layers of the skin, nails, or hair. Such infections may present as a mild inflammation, and can cause alternating remissions and eruptions of a gradually extending, scaling, raised lesion. Yeast infections, such as candidiasis and oral candidiasis (thrush), are usually localized to the skin and mucous membranes, with the symptoms varying depending on the site of infection. In many instances, such infections appear as erythematous, often itchy, exudative patches in the groin, axillas, umbilicus, between toes, and on finger-webs. Oral thrush involves an inflamed tongue or buccal mucosa, typically accompanied by white patches of exudate. Chronic mucocutaneous candidiasis is manifested in the form of red, pustular, crusted, thickened lesions on the forehead or nose.

Itraconazole Chemistry and Uses

Itraconazole is a broad-spectrum antifungal agent developed for oral, parenteral and topical use, and is disclosed in U.S. Pat. No. 4,267,179. Itraconazole is a synthetic triazole derivative that disrupts the synthesis of ergosterol, the primary sterol of fungal cell membranes. This disruption appears to result in increased permeability and leakage of intracellular content, and at high concentration, cellular internal organelles involute, peroxisomes increase, and necrosis occurs.

As set forth in the USP Dictionary of Drug Names and USAN, itraconazole is defined as 4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one, or alternatively, as (±)-1-sec-butyl-4-[p-[4-[p-[[(2R*,4S*)-2-(2,4-dichlorophenyl)-2-(H-1,2,4-triazol-1-ylmethyl)-1,3-dioxolan-4-yl]methoxy]phenyl]-1-piperazinyl]phenyl]-Δ²-1,2,4-triazolin-5-one. There are three asymmetric carbons in itraconazole: one in the sec-butyl side chain on the triazolone and two in the dioxolane ring. As a result, eight possible stereoisomers of itraconazole exist: (R,R,R), (S,S,S), (R,R,S), (S,S,R), (R,S,S), (R,S,R), (S,R,S), and (S,R,R).

(±)Cis-Itraconazole comprises a mixture of only those isomers that describe a “cis” relationship in the dioxolane ring, i.e., the (1H-1,2,4-triazol-1-ylmethyl) moiety and the substituted phenoxy moiety are located on the same side of a plane defined by the 1,3-dioxolane ring. By convention, the first represented chiral center is at the C-2 position of the dioxolane ring, the second is at the C-4 position of the dioxolane ring, and the third is in the sec-butyl group. Hence, (O)cis-itraconazole is a mixture of (R,S,S), (R,S,R), (S,R,S) and (S,R,R) isomers.

The four possible stereoisomeric cis forms of itraconazole, and diastereomeric pairs thereof, are described in more detail in U.S. Pat. Nos. 5,474,997 and 5,998,413. In general, the individual stereoisomeric forms of cis-itraconazole have antifungal properties, and contribute to the overall activity of (O)cis-itraconazole.

(±)Cis-Itraconazole free base is only very sparingly soluble in water, and thus it is extremely difficult to prepare effective pharmaceutical compositions containing the same. A number of means have been used to increase the solubility of itraconazole free base, including complexing or co-formulation with cyclodextrins or derivatives thereof, as described in U.S. Pat. No. 4,764,604, U.S. Pat. No. 5,998,413, and U.S. Pat. No. 5,707,975, and coating beads with a film comprising a hydrophilic polymer and itraconazole, as described in U.S. Pat. No. 5,633,015.

Another approach to increase solubility of itraconazole focuses on preparation of the stereoisomers of cis-itraconazole, and in particular (2R,4S) itraconazole, which may comprise a mixture of two diastereomers ((R,S,S) and (R,S,R)), as described in U.S. Pat. Nos. 5,414,997 and 5,998,413.

Commercially available itraconazole (SPORANOX® brand (O)cis-itraconazole, Janssen Pharmaceutica Products, L.P., Titusville, N.J., U.S.A.) is a free base and a racemic mixture of the cis isomer in the dioxolane ring and is represented by structural formula (I):

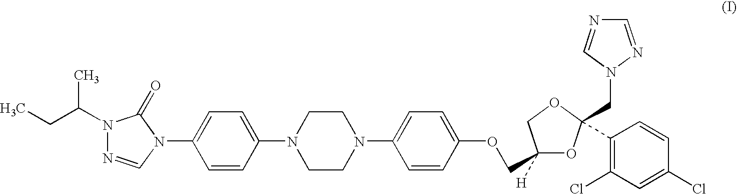

SPORANOX® has been approved for use as an antifungal agent for treating immunocompromised and non-immunocompromised patients having: blastomycosis (pulmonary and extrapulmonary); histoplasmosis, including chronic cavitary pulmonary disease and disseminated non-meningeal histoplasmosis; and aspergillosis. In addition, in non-immunocompromised patients, it has been approved for treatment of onychomycosis. See generally, Physician's Desk Reference, 56^{th} ed. (2002). The compound has also been investigated for use in coccidioidomycosis, cryptococcosis, dermatophyte, and candidiasis infections.

Adverse effects associated with the administration of (O)cis-itraconazole free base include nausea, vomiting, anorexia, headache, dizziness, hepatotoxicity, and inhibition of drug metabolism in the liver, leading to numerous, clinically significant, adverse drug interactions. See, Physician's Desk Reference, 56^{th} ed. (2002); Honig et al., J. Clin. Pharmacol. 33:1201-1206 (1993) (terfenadine interaction); Gascon and Dayer, Eur. J. Clin. Pharmacol., 41:573-578 (1991) (midazolam interaction); and Neuvonen et al., Clin. Pharmacol Therap., 60:54-61 (1996) (lovastatin interaction). Reactions associated with hypersensitivity, such as urticaria and serum liver enzymes elevation, are also associated with the administration of the drug. A more serious, though less common, adverse effect is hepatotoxicity. See, e.g., Lavrijsen et al., Lancet, 340:251-252 (1992).

In addition, as discussed herein, cis-itraconazole free base is only very sparingly soluble in water. Thus, due to its relative non-polarity and insolubility, itraconazole free base suffers from two other drawbacks: it cannot be readily formulated in parenteral solution, and it does not effectively penetrate the blood-brain barrier. The latter problem is exacerbated by drug interactions, such as one observed between itraconazole free base and valproate, as described in VIIIa et al., Rev. Inst. Med. Trop., Sao Paulo, pp. 231-234 (July-August 2000), which is incorporated by reference herein in its entirety. In another case of CNS fungal infection, extremely high doses of itraconazole free base were used to treat residual aspergillus infection, as reported by Imai et al., Intern. Med., 38(10):829-832 (1999), which is incorporated by reference herein in its entirety. As a result, numerous therapeutic indications that require rapid achievement of effective blood levels or access to the CNS are difficult to treat or beyond treatment with itraconazole free base.

Furthermore, the emergence of antifungal resistance (e.g., in Aspergillus fumigatus isolates as described by Dannaoui et al., J. Antimicrob. Chemother., 47:333-340 (2001), which is incorporated by reference herein in its entirety) presents an added challenge to the efficacy of itraconazole free base. For those strains of fungi that show resistance, high and relatively constant levels of itraconazole free base must be produced in the target organs of infected patients.

Over the years, a number of formulation routes have been used in order to enhance the adsorption and bioavailability of itraconazole. For example, the currently marketed SPORANOX® solid dosage capsule form of itraconazole free base utilizes sugar-based beads coated with a hydrophilic polymer and an amorphous film of itraconazole. See Physicians Desk Reference, 56^{th} ed., pp. 1800-1804 (2002); and U.S. Pat. No. 5,633,015. This dosage form requires up to two capsules three times daily depending on the condition being treated.

Even with the various formulation routes, the dosage amounts and dose frequency for itraconazole can be burdensome to patients. In addition, administration of existing dosage forms of itraconazole have shown significant variability in bioavailability and adsorption, which likely results from food effects. See, Physician's Desk Reference, 56^{th} ed., pp. 1800-1804 (2002). Thus, it would be desirable to increase bioavailability and adsorption and decrease the per-dose pill count and decrease dosing frequency (e.g., twice a day to once a day) associated with administration of itraconazole in order to provide an improvement over current therapy, particularly with regard to patient compliance, convenience, ease of ingestion, especially with regard to immunocompromized polypharmacy patients (e.g., AIDS or cancer patients).

Posaconazole and Saperconazole Chemistry and Uses

Other related conazoles have also been discovered and used as antifungals. Two of these conazoles that are closely structurally related to itraconazole are posaconazole and saperconazole. Posaconazole (CAS Registry Number: 171228-49-2; CAS Name: 2,5-Anhydro-1,3,4-trideoxy-2-C-(2,4-difluorophenyl)-4-[[4-[4-[4-[1-[(1S,2S)-1-ethyl-2-hydroxypropyl]-1,5-dihydro-5-oxo-4H-1,2,4-triazol-4-yl]phenyl]-1-piperazinyl]phenoxy]methyl]-1-(1H-1,2,4-triazol-1-yl)-D-threo-pentitol; Additional Name: (3R-cis)-4-[4-[4-[4-[5-(2,4-difluorophenyl)-5-(1,2,4-triazol-1-ylmethyl)tetrahydrofuran-3-ylmethoxy]phenyl]piperazin-1-yl]phenyl]-2-[1(S)-ethyl-2(S)-hydroxypropyl]-3,4-dihydro-2H-1,2,4-triazol-3-one) is represented by structural formula (II):

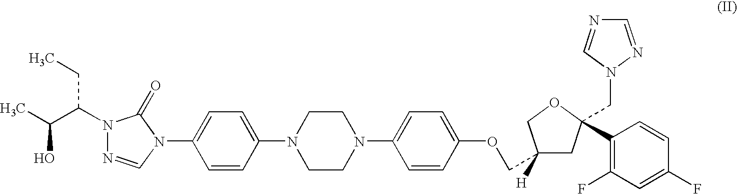

Saperconazole (CAS Registry Number: 110588-57-3; CAS Name: 4-[4-[4-[4-[[2-(2,4-Difluorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one; Additional Name: (±)-1-sec-butyl-4-[p-[4-[p-[[(2R*,4S*)-2-(2,4-difluorophenyl)-2-(1H-1,2,4-triazol-1-ylmethyl)-1,3-dioxolan-4-yl]methoxy]phenyl]-1-piperazinyl]phenyl]-Δ²-1,2,4-triazolin-5-one) is represented by structural formula (III):

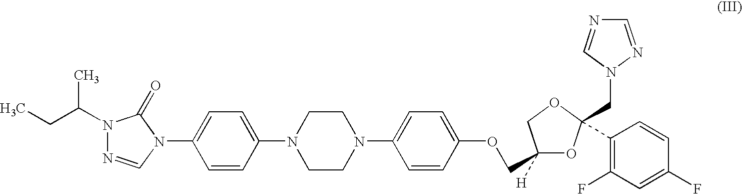

Consequently, there is a need for soluble forms of conazoles including cis itraconazole, posaconazole and saperconazole that can be readily formulated for use in various modes of administration, including parenteral and oral administration.

### SUMMARY OF THE INVENTION

The invention provides novel soluble crystalline forms of conazoles including cis-itraconazole, posaconazole or saperconazole comprising the reaction product of the conazole and an organic acid or an inorganic acid including salts, co-crystals, solvates, hydrates and multicomponent crystal systems having three or more components (including saperconazole). In one embodiment, the soluble crystalline form of the conazole, comprises the reaction product of the conazole, e.g., cis-itraconazole, posaconazole or saperconazole, and a dicarboxylic acid or a carboxylic acid. The invention includes novel soluble conazole (e.g., cis-itraconazole, posaconazole or saperconazole) salts, co-crystals, solvates, hydrates, and polymorphs.

In one embodiment, the invention provides a soluble, multicomponent crystalline system comprising:

(a) the reaction product of a conazole and an organic acid or an inorganic acid; and

(b) one or more organic or inorganic solvents wherein the organic solvent is present in the system in either a stoichiometric or non-stoichiometric ratio relative to the organic salt or a second reaction product of a conazole and an organic acid or an inorganic acid.

In a further embodiment, the multicomponent crystalline system is a co-crystal comprising a co-crystal former and a conazole.

In a further embodiment, the reaction product is a salt.

In a further embodiment, the reaction product is a co-crystal.

In a further embodiment, the first reaction product is a salt and the second reaction product is a co-crystal.

In a further embodiment, the system comprises a first reaction product, a second reaction product and a solvent.

In a further embodiment, the invention provides for a co-crystal comprising a co-crystal former and a conazole free base or a co-crystal former and a conazole salt. Either co-crystal form may further comprise a solvent as provided for herein.

Further, in several embodiments of the invention, the novel soluble crystalline form of cis-itraconazole is characterized by an endothermic transition temperature, a Raman spectrum, a crystal morphology or by selected peaks of a powder X-ray diffraction pattern expressed in terms of 2 theta angles, wherein the X-ray powder diffraction patterns comprise the 2 theta angle values listed herein.

The invention also provides pharmaceutical compositions comprising, and processes for making, conazole (e.g., cis itraconazole posaconazole or saperconazole) crystalline forms including salts, co-crystals, solvates, etc. Methods of using such compositions for the treatment or prevention of systemic and local fungal, yeast, and dermatophyte infections are also provided.

Compounds of the invention include, but are not limited to, soluble crystalline forms of conazoles including: saperconazole D-, L-, and D,L-tartaric acid co-crystal, saperconazole succinic acid co-crystal, saperconazole fumaric acid co-crystal, saperconazole L-malic acid co-crystal, saperconazole glutaric acid co-crystal, and saperconazole mesylate salt. Soluble crystalline forms of conazoles (e.g., saperconazole) of the invention include dicarboxylic acid salts, dicarboxylic acid co-crystals, and hydrochloric acid salt co-crystals. Other soluble crystalline forms of cis-itraconazole, posaconazole or saperconazole include hydrochloric acid, phosphoric acid, sulfuric acid or benzenesulfonic acid salts and co-crystals. Other compounds of the invention include crystalline forms of an alcohol solvate (e.g., ethanol, methanol, propylene glycol, propanol, etc.) or dioxane solvate, or a conazole (e.g., a cis-itraconazole, posaconazole or saperconazole) co-crystal such as tartaric acid co-crystal, fumaric acid co-crystal, malic acid co-crystal, maleic acid co-crystal, adipic acid co-crystal, di-mesylate, and succinic acid co-crystal. Other soluble crystalline forms of cis-itraconazole, posaconazole or saperconazole include DL-tartaric acid, succinic acid, L-malic acid, fumaric acid, and glutaric acid co-crystals. Other soluble crystalline forms of cis-itraconazole, posaconazole or saperconazole include methanesulfonic acid salts. In one embodiment the co-crystal comprises a co-crystal former and a conazole salt. In another embodiment, the co-crystal further comprises a solvent.

The invention further provides methods of treating or preventing local and systemic fungal, yeast, and dermatophyte infections in a patient by administration of therapeutically or prophylactically effective amounts of soluble crystalline forms of a conazole such as cis-itraconazole, posaconazole or saperconazole, comprising the reaction product of a conazole such as cis-itraconazole, posaconazole or saperconazole, and an organic acid or an inorganic acid. Many pharmaceutical dosage forms of the invention comprise therapeutically or prophylactically effective amounts of soluble crystalline forms of a conazole (e.g., cis-itraconazole, posaconazole or saperconazole) comprising the reaction product of cis-itraconazole, posaconazole or saperconazole and an organic acid or an inorganic acid.

The invention also provides medicaments comprising, and processes for making, conazole (e.g., cis itraconazole posaconazole or saperconazole) crystalline forms including salts, co-crystals, solvates, etc. Methods of using such medicaments for the treatment or prevention of systemic and local fungal, yeast, and dermatophyte infections are also provided. Many medicaments of the invention comprise therapeutically or prophylactically effective amounts of soluble crystalline forms of a conazole (e.g., cis-itraconazole, posaconazole or saperconazole) comprising the reaction product of cis-itraconazole, posaconazole or saperconazole and an organic acid or an inorganic acid.

### BRIEF DESCRIPTION OF THE DRAWINGS

FIG. 1—A schematic of a conazole co-crystal comprising a trimer consisting of a co-crystal former between two antiparallel conazole molecules;

FIG. 2—DSC thermogram of saperconazole;

FIG. 3—TGA thermogram of saperconazole;

FIG. 4—Raman spectrum of saperconazole;

FIG. 5—PXRD diffractogram of saperconazole;

FIG. 6—DSC thermogram of saperconazole:DL-tartaric acid co-crystal;

FIG. 7—TGA thermogram of saperconazole:DL-tartaric acid co-crystal;

FIG. 8—PXRD diffractogram of saperconazole:DL-tartaric acid co-crystal;

FIG. 9—DSC thermogram of saperconazole:succinic acid co-crystal;

FIG. 10—TGA thermogram of saperconazole:succinic acid co-crystal (first synthesis);

FIG. 11—PXRD diffractogram of saperconazole:succinic acid co-crystal (first synthesis);

FIG. 12—TGA thermogram of saperconazole:succinic acid co-crystal (second synthesis);

FIG. 13—TGA thermogram of saperconazole:succinic acid co-crystal (top, first synthesis; bottom, second synthesis);

FIG. 14—PXRD diffractogram of saperconazole:succinic acid co-crystal (second synthesis);

FIG. 15—DSC thermogram of saperconazole:L-malic acid co-crystal;

FIG. 16—TGA thermogram of saperconazole:L-malic acid co-crystal;

FIG. 17—PXRD diffractogram of saperconazole:L-malic acid co-crystal;

FIG. 18—DSC thermogram of saperconazole:fumaric acid co-crystal;

FIG. 19—TGA thermogram of saperconazole:fumaric acid co-crystal;

FIG. 20—PXRD diffractogram of saperconazole:fumaric acid co-crystal;

FIG. 21—PXRD diffractogram of saperconazole:glutaric acid co-crystal;

FIG. 22—DSC thermogram of saperconazole mesylate salt;

FIG. 23—TGA thermogram of saperconazole mesylate salt; and

FIG. 24—PXRD diffractogram of saperconazole mesylate salt.

### DETAILED DESCRIPTION OF THE INVENTION

As used herein, the following terms have the following respective meanings.

As used herein, the term “solvate” is a complex of variable stoichiometry formed by a solute (either cis-itraconazole, posaconazole or saperconazole or salts, co-crystals, hydrates, or polymorphs of cis-itraconazole, posaconazole or saperconazole) and a liquid at room temperature, including an alcohol, such as methanol or ethanol, or dioxane.

“Carboxylic acids” include, but are not limited to, formic, acetic, propionic, butyric, isobutyric, valeric, isovaleric, pivalic, caproic, caprylic, capric, lauric, myristic, palmitic, stearic, acrylic, crotonic, benzoic, cinnamic, and salicylic acids.

“Dicarboxylic acid” means a compound of formula (IV): 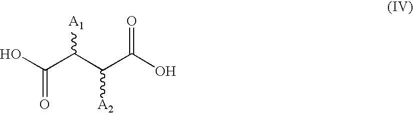
wherein A₁ and A₂ are each independently H, OH, Cl, Br, I, substituted or unsubstituted C₁₋₆ alkyl, substituted or unsubstituted aryl or A₁ and A₂ taken together represent a double bond as well as stereochemically pure D or L salts of a compound of formula (IV). Examples of the dicarboxylic acid of formula (IV) include, but are not limited to, succinic acid, maleic acid, tartaric acid, malic acid or fumaric acid. It should be recognized that additional dicarboxylic acids such as, but not limited to, malonic acid and adipic acid are distinct embodiments of the invention although they fall outside the scope of formula (IV).

“Organic or inorganic acids” include, but are not limited to, carboxylic acids, dicarboxylic acids, hydrochloric acid, phosphoric acid, sulfuric acid, benzenesulfonic acid, methanesulfonic acid, and, in general terms, any acidic species that will form a thermodynamically stable crystalline (salt) form upon reaction with the free base cis-itraconazole, posaconazole or saperconazole.

The term “co-crystal” as used herein means a crystalline material comprised of two or more unique solids at room temperature, each containing distinctive physical characteristics, such as structure, melting point and heats of fusion, with the exception that, if specifically stated, the API may be a liquid at room temperature. The co-crystals of the present invention comprise a co-crystal former H-bonded to an API. The co-crystal former may be H-bonded directly to the API or may be H-bonded to an additional molecule which is bound to the API. The additional molecule may be H-bonded to the API or bound ionically or covalently to the API. The additional molecule could also be a different API. Solvates of API compounds that do not further comprise a co-crystal former are not co-crystals according to the present invention. The co-crystals may however, include one or more solvate molecules in the crystalline lattice. That is, solvates of co-crystals, or a co-crystal further comprising a solvent or compound that is a liquid at room temperature, is included in the present invention, but crystalline material comprised of only one solid and one or more liquids (at room temperature) are not included in the present invention, with the previously noted exception of specifically stated liquid APIs. The co-crystals may also be a co-crystal between a co-crystal former and a salt of an API, but the API and the co-crystal former of the present invention are constructed or bonded together through hydrogen bonds. Other modes of molecular recognition may also be present including, pi-stacking, guest-host complexation and van der Waals interactions. Of the interactions listed above, hydrogen-bonding is the dominant interaction in the formation of the co-crystal, (and a required interaction according to the present invention) whereby a non-covalent bond is formed between a hydrogen bond donor of one of the moieties and a hydrogen bond acceptor of the other. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads. For purposes of the present invention, the chemical and physical properties of an API in the form of a co-crystal may be compared to a reference compound that is the same API in a different form. The reference compound may be specified as a free form, or more specifically, a free acid, free base, or zwitterion; a salt, or more specifically for example, an inorganic base addition salt such as sodium, potassium, lithium, calcium, magnesium, ammonium, aluminum salts or organic base addition salts, or an inorganic acid addition salts such as HBr, HCl, sulfuric, nitric, or phosphoric acid addition salts or an organic acid addition salt such as acetic, propionic, pyruvic, malanic, succinic, malic, maleic, fumaric, tartaric, citric, benzoic, methanesulfonic, ethanesulforic, stearic or lactic acid addition salt; an anhydrate or hydrate of a free form or salt, or more specifically, for example, a hemihydrate, monohydrate, dihydrate, trihydrate, quadrahydrate, pentahydrate, sesquihydrate; or a solvate of a free form or salt. For example, the reference compound for an API in salt form co-crystallized with a co-crystal former can be the API salt form. Similarly, the reference compound for a free acid API co-crystallized with a co-crystal former can be the free acid API. The reference compound may also be specified as crystalline or amorphous.

“Soluble crystalline forms” or “multicomponent crystalline systems” encompass crystalline species including salts, hydrates, solvates, co-crystals, and polymorphs that are soluble in aqueous media at values greater than 5 micrograms/mL, greater than 10 micrograms/mL, greater than 20 micrograms/mL, greater than 30 micrograms/mL, greater than 40 micrograms/mL, greater than 50 micrograms/mL, and greater than 100 micrograms/mL in a solution with a pH of about 1. It is understood that while polymorphs are not multicomponent crystalline systems, they can be considered soluble crystalline forms, if applicable. Soluble multicomponent crystalline systems can comprise: (a) an organic compound comprising the reaction product of cis-itraconazole, posaconazole or saperconazole and an organic acid or an inorganic acid; and (b) one or more organic solvents, wherein the organic solvent is present in either a stoichiometric or non-stoichiometric ratio relative to the organic salt.

“Organic solvent” includes, but not is limited to, 1,4-dioxane (dioxane), 1,2-dichloroethane, dimethoxyethane, diethylene glycol dimethyl ether, tetrahydrofuran, diisopropyl ether, hydrocarbons such as hexane, heptane, cyclohexane, toluene, or xylene, alcohols such as methanol, ethanol, 1-propanol, 2-propanol, 1-butanol, 2-butanol, tert-butanol or ethylene glycol, ketones such as methyl ethyl ketone or isobutyl methyl ketone, amides such as dimethylformamide, dimethylacetamide, N-methylpyrrolidone, and mixtures thereof.

The term “anomer” as used herein means one of a pair of isomers of a cyclic compound resulting from creation of a new point of symmetry when a rearrangement of atoms occurs at an aldehyde or ketone position.

“Alkyl” means a straight chain or branched, saturated or unsaturated alkyl, cyclic or non-cyclic hydrocarbon having from 1 to 10 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (also referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cycloalkyls are also referred to herein as “carbocyclic” rings systems, and include bi- and tri-cyclic ring systems having from 8 to 14 carbon atoms such as a cycloalkyl (such as cyclopentane or cyclohexane) fused to one or more aromatic (such as phenyl) or non-aromatic (such as cyclohexane) carbocyclic rings.

As used herein, the term “aryl” means a carbocyclic or heterocyclic aromatic group containing from 5 to 10 ring atoms. The ring atoms of a carbocyclic aromatic group are all carbon atoms, and include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. A carbocyclic aromatic group can be unsubstituted or substituted. For example, the carbocyclic aromatic group is a phenyl group. The ring atoms of a heterocyclic aromatic group contains at least one heteroatom, for example 1 to 3 heteroatoms, independently selected from nitrogen, oxygen, and sulfur. Illustrative examples of heterocyclic aromatic groups include, but are not limited to, pyridinyl, pyridazinyl, pyrimidyl, pyrazyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isoxazolyl, thiazolyl, furyl, phienyl, isoxazolyl, indolyl, oxetanyl, azepinyl, piperazinyl, morpholinyl, dioxanyl, thietanyl and oxazolyl. A heterocyclic aromatic group can be unsubstituted or substituted. For example, a heterocyclic aromatic is a monocyclic ring, wherein the ring comprises 2 to 5 carbon atoms and 1 to 3 heteroatoms.

The term “substituted” as used herein means any of the above groups (i.e., aryl or alkyl) wherein at least one hydrogen atom is replaced with a substituent. In the case of a keto substituent (C(═O)) two hydrogen atoms are replaced. Substituents include halogen, hydroxy, alkyl, aryl, arylalkyl, heterocycle or heterocyclealkyl.

As used herein, the term “cis-itraconazole” refers to (±)cis-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one, its four stereoisomers (+)-[2R-[2α,4α, 4(R)]-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one (also referred to as the (R,S,R) stereoisomer), (+)-[2R-[2α,4α,4(S)]-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one (also referred to as the (R,S,S) stereoisomer), (−)-[2S-[2α,4α,4(R)]-4-[4-[4-[4-[[2-(2,4-dichlorophenyl)-2-(1H-1,2,4-triazol-1-ylmethyl)-1,3-dioxolan-4-yl]methoxy]phenyl]1-piperazinyl]phenyl]-2,4-dihydro-2-(1-methylpropyl)-3H-1,2,4-triazol-3-one (also referred to as the (S,R,R) stereoisomer), and (−)-[2S-[2α,4α,4(S)]-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one (also referred to as the (S,R,S) stereoisomer), and diastereomeric pairs thereof. As used herein, the terms “cis-itraconazole” and “itraconazole” are used interchangeably throughout, and both are defined as stated above.

As used herein, the term “cis-itraconazole, posaconazole or saperconazole tartaric acid co-crystal” refers to novel soluble crystalline forms of cis-itraconazole, posaconazole or saperconazole-DL-tartaric acid co-crystal, cis-itraconazole, posaconazole or saperconazole-L-tartaric acid co-crystal, and cis-itraconazole, posaconazole or saperconazole-D-tartaric acid co-crystal. Similarly, where appropriate the other co-crystals refer to racemic or “DL” co-crystals unless otherwise indicated.

As used herein, the terms “stereoisomer” or “stereoisomeric form” means compounds having a stereoisomeric purity of at least 90%, at least 95%, or up to a stereoisomeric purity of 100% by weight, for example compounds having a stereoisomeric purity of at least 97% up to a stereoisomeric purity of 100%, such as a stereoisomeric purity of at least 99% up to a stereoisomeric purity of 100% by weight, said weight percent based upon the total weight of the desired stereoisomers of the compound.

As used herein, the term “diastereomeric pair” refers to a mixture of two stereoisomers of cis-itraconazole, and in particular, either 1) a mixture of (+)-[2R-[2α,4α,4(R)]-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one (the (R,S,R) stereoisomer) and (+)-[2R-[2α,4α,4(S)]-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one (the (R,S,S) stereoisomer), or 2) a mixture of (−)-[2S-[2α,4α,4(R)]-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one (the (S,R,R) stereoisomer), and (−)-[2S-[2α,4α,4(S)]-4-[4-[4-[4-[[2-(2,4-dichlorophenyl)-2-(1H-1,2,4-triazol-1-ylmethyl)-1,3-dioxolan-4-yl]methoxy]phenyl]-1-piperazinyl]phenyl]-2,4-dihydro-2-(1-methylpropyl)-3H-1,2,4-triazol-3-one (the (S,R,S) stereoisomer). In another embodiment, the mixture is in the range of a 47:53 to a 53:47 mixture by weight, a 48:52 to a 52:48 mixture by weight, or the mixture is a 50:50 mixture by weight.

As used herein, the term “adjunctively administered” refers to the administration of one or more compounds or active ingredients in addition to a pharmaceutically acceptable salt or co-crystal of cis-itraconazole, posaconazole or saperconazole, or a hydrate, solvate or polymorph thereof, either simultaneously with the same or at intervals prior to, during, or following administration of the pharmaceutically acceptable salt or co-crystal of cis-itraconazole, posaconazole or saperconazole to achieve the desired therapeutic or prophylactic effect.

As used herein, the term “pharmaceutically acceptable salt” refers to a salt prepared from pharmacologically acceptable anions, such as hydrochloride, phosphate, formate, adipate, succinate, fumarate, malate, tartrate, malonate, maleate, mesylate and benzenesulfonate. For example, anions are tartrate, benzenesulfonate, malate and succinate, hydrobromide, bitartrate, para-toluenesulfonate, glycolate, glucuronate, mucate, gentisate, isonicotinate, saccharate, acid phosphate, hydroiodide, nitrate, sulfate, bisulfate, acetate, propionate, camphorsulfonate, gluconate, isothionate, lactate, furoate, glutamate, ascorbate, benzoate, anthranilate, salicylate, phentylacetate, mandelate, embonate (pamoate), methanesulfonate, ethanesulfonate, pantothenate, stearate, sulfanilate, alginate, p-toluenesulfonate, mesylate, and galacturonate.

As used herein, the term “method of treating or preventing local and systemic fungal, yeast and dermatophyte infections” means prevention of, or relief from local and systemic fungal, yeast and dermatophyte infections, or one or more symptoms thereof. Local and systemic fungal, yeast and dermatophyte infections include, but are not limited to blastomycosis, aspergillosis, histoplasmosis, onychomycosis, coccidioidomycosis, paracoccidioidomycosis, cryptococcosis, dermatophyte, and candidiasis infections.

The term “conazole” refers to compounds comprising a substituted or unsubstituted 1,2,4-triazol group or a substituted or unsubstituted 1-H-imidazole group. Conazoles can further be specified as having antifungal activity and useful as an active pharmaceutical ingredient. Conazoles can further be defined as comprising both a 1,2,4-triazol and a 1-H-imidazole group and, optionally, having antifungal activity.

The reference compounds to the present invention herein can refer to the free base neutral form of the appropriate conazole, either crystalline or amorphous, or SPORANOX®.

Further more specific compounds of the present invention include salts, co-crystals, multicomponent systems, solvates, hydrates and polymorphs of itraconazole, posaconazole, saperconazole and derivatives thereof:

Itraconazole

This invention is concerned in part with 1H-imidazoles and 1H-1,2,4-triazoles having the formula (V): 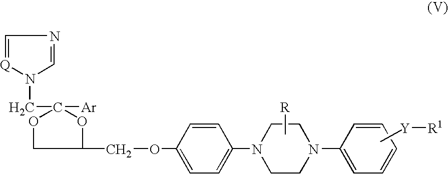
and the stereochemically isomeric forms thereof, wherein
Q is N or CH;
Ar is aryl;
R is hydrogen or C₁₋₆ alkyl; and
Y—R¹ is a radical having the formula (VI): 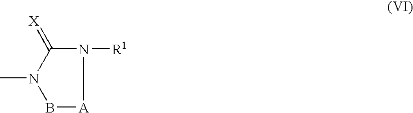
or a radical having the formula (VII): 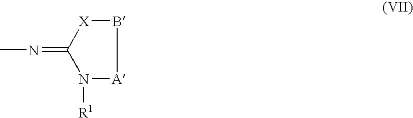
wherein R¹ is tetrahydrofuranyl C₁₋₆ alkyl; or C₁₋₆ alkyl, C₃₋₆ cycloalkyl, arylC₁₋₆ alkyl or (C₃₋₆ cycloalkyl) C₁₋₆ alkyl all substituted on the C₁₋₆ alkyl and/or C₃₋₆ cycloalkyl moiety with oxo, thioxo or with one or two radicals of formula -Z-R^{1-a};
said Z being O or S;
said R^{1-a} being hydrogen, C₁₋₆ alkyl, aryl, C₃₋₆ cycloalkyl or tetrahydro 2H-pyran-2-yl;
or where R¹ is substituted with two-Z-R^{1-a} radicals, the two —R^{1-a} radicals, taken together, may form a bivalent radical of formula —CH₂—, —CH(CH₃)—, —C(CH₃)₂—, —CH₂—CH₂—, —CH(CH₃)—CH₂— or —CH₂—CH₂—CH₂—;
X is O, S or NR²;
said R² being hydrogen or C₁₋₆ alkyl;
A is C═O, NR³ or methylene, optionally substituted with up to two radicals selected from the group consisting of C₁₋₆ alkyl and aryl;
said R³ being hydrogen or C₁₋₆ alkyl;
B is C═O or methylene optionally substituted with up to two radicals selected from the group consisting of C₁₋₆ alkyl and C₁₋₆ alkyloxy;
or A and B, taken together, form a bivalent radical of formula (VIII):
—N═CH—; (VIII)
A′ and B′ independently having the same meaning of A and B respectively, or A′ and B′, taken together, form a bivalent radical of formula (VIII) or (IX):
—N═CH— or (VIII)
—CH═CH—; (IX)
wherein the nitrogen atom in the bivalent radical (VIII) is connected to NR¹; wherein one hydrogen in said radical (VIII) and up to two hydrogens in radical (IX) may be replaced by a C₁₋₆ alkyl radical; provided that

(i) when Y—R¹ is a radical of formula (VI) wherein -A-B— is other than a bivalent radical of formula (VIII), then R¹ is other than C₁₋₆ alkyl substituted with C₁₋₆ alkyloxy;

(ii) when Y—R¹ is a radical of formula (VII) then R′ is other than C₁₋₆ alkyl substituted with C₁₋₆ alkyloxy;

wherein aryl is phenyl or substituted phenyl, said substituted phenyl having from 1 to 3 substituents each independently selected from the group consisting of halo, C₁₋₆ alkyl, C₁₋₆ alkyloxy, nitro, amino and trifluoromethyl, provided that trinitrophenyl is excluded.

In the foregoing definitions the term “halo” is generic to fluoro, chloro, bromo and iodo; the term “C₁₋₆ alkyl” is meant to include straight and branched hydrocarbon radicals having from 1 to 6 carbon atoms such as for example, methyl, ethyl, 1-methylethyl, 1,1-dimethylethyl, propyl, 1-methylpropyl, 2-methylpropyl, butyl, pentyl, hexyl and the like; “C₃₋₆ cycloalkyl” embraces cyclopropyl, cyclobutyl, cyclopentyl and cyclohexyl.

The compounds of formula (V) may contain in their structure a tautomeric system and consequently these compounds can be present in each of their tautomeric forms.

Compounds within the invention are those wherein Y—R¹ is a radical of formula (VI) or (VII), wherein X, A, B, A′, B′ and R′ are as described hereinabove, provided that A′ and B′, taken together, do not form a radical of formula (VIII) or (IX).

More specifically, compounds within the invention are those compounds wherein Y—R¹ is a radical of formula (VI).

Further specific compounds within the invention are those compounds wherein

X is 0;

A and B are independently C═O or methylene. optionally substituted with up to two C₁₋₆ alkyl radicals, or A and B, taken together, form a bivalent radical of formula (VIII) wherein the hydrogen atom may be replaced by a C₁₋₆ alkyl radical; and R¹ is tetrahydrofuranyl C₁₋₆ alkyl, or C₃₋₆ cycloalkyl substituted with oxo or hydroxy, or C₁₋₆ alkyl or aryl C₁₋₆ alkyl both substituted on the C₁₋₆ alkyl moiety with oxo or with one or two radicals of formula —O—R^{1-a}, or where R¹ is substituted with two —O—R^{1-a} radicals, the two —R^{1-a} radicals, taken together, may form a bivalent radical of formula —C(CH₃)₂— or —CH₂—.

More specifically, compounds within the invention are those wherein R¹ is C₃₋₆ cycloalkyl substituted with oxo or hydroxy, or C₁₋₆ alkyl or aryl C₁₋₆ alkyl both substituted on the C₁₋₆ alkyl moiety with oxo or with one or two hydroxy or C₁₋₆ alkyloxy radicals.

More specifically, compounds within the invention are those compounds wherein Ar is phenyl substituted with two halo atoms: R is hydrogen: A is C(CH₃)₂ or CH₂, B is CH₂ or C═O, or A and B, taken together, form a radical (VIII) wherein the hydrogen atom may be replaced by a methyl radical; and R¹ is C₆ alkyl substituted with oxo or hydroxy.

Saperconazole

This invention is further concerned in part with 1H-imidazoles and 1H-1,2,4-triazoles having the formula (X):

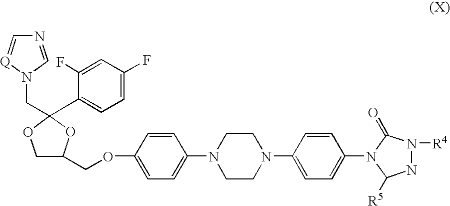

and the stereochemically isomeric forms thereof, wherein

Q is N or CH;

R⁴ is hydrogen, C₁₋₆ alkyl or arylC₁₋₆ alkyl; and

R⁵ is hydrogen, C₁₋₆ alkyl or arylC₁₋₆ alkyl;

wherein aryl is phenyl optionally substituted with up to 3 substituents each independently selected from halo, C₁₋₆ alkyl, C₁₋₆ alkyloxy and trifluoromethyl.

In the foregoing definitions the term “halo” is generic to fluoro, chloro, bromo and iodo and the term “C₁₋₆ alkyl” is meant to include straight and branched hydrocarbon radicals having from 1 to 6 carbon atoms such as for example, methyl, ethyl, propyl, 1-methylethyl, 1,1-dimethylethyl, 1-methylpropyl, 2-methylpropyl, butyl, pentyl, hexyl and the like.

The compounds of formula (X) wherein R⁴ is hydrogen contain in their structure a tautomeric system and consequently these compounds can be present in each of their tautomeric forms, both of which are intended to be included within the scope of the present invention.

Compounds within the present invention are those compounds of formula (X) wherein R⁴ and R⁵ independently are hydrogen or C₁₋₆ alkyl.

More specifically, compounds are the above compounds wherein R⁵ is hydrogen and R⁴ is C₁₋₆ alkyl.

More specifically, compounds are the above compounds wherein the substituents on the dioxolane moiety have a cis configuration.

A particular subgroup of the compounds of formula (X) comprises the compounds above, where Q is nitrogen.

More specific compounds above are selected from the group consisting of cis-4-[4-[4-[4-[[2-(2,4-difluorophenyl)-2-(1H-1,2,4-triazol-1-yl-methyl)-1,3-dioxolan-4-yl]methoxy]phenyl]-1-piperazinyl]phenyl]-2,4-dihydro-2-(1-methylpropyl)-3H-1,2,4-triazol-3-one and cis-4-[4-[4-[4-[[2-(2,4-difluorophenyl)-2-(1H-1,2,4-triazol-1-ylmethyl)-1,3-dioxolan-4-yl]methoxy]phenyl]-1-piperazinyl]phenyl]-2-(1,2-dimethylpropyl)-2,4-dihydro-3H-1,2,4-triazol-3-one.

Posaconazole

The present invention provides compounds represented by formula (XI):

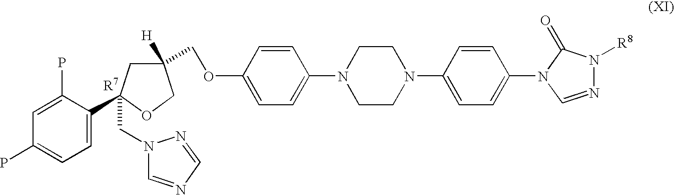

wherein P is independently both F or both Cl or one X is independently F and the other is independently Cl;

R⁸ is a straight or branched chain (C₃ to C₈) alkyl group substituted by one or two hydroxy moieties or stereoisomers thereof or by one or two groups convertible in vivo into hydroxy moieties or an ester or ether thereof.

In an aspect of the present invention, there is provided compounds represented by formula (XII) 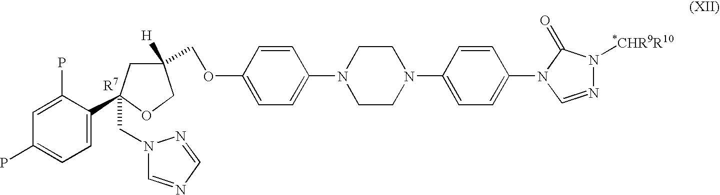
wherein P is independently both F or both Cl or one P is independently F and the other is independently Cl;

wherein R⁹ is H or (C₁-C₃) alkyl and R¹⁰ is (C₁-C₃) alkyl substituted by one hydroxy moiety or by a group convertible in vivo into a hydroxy moiety and the carbon with the asterisk (*) has the R or S absolute configuration; an ester or ether thereof.

In another aspect, the present invention provides a compound represented by formula (XIII): 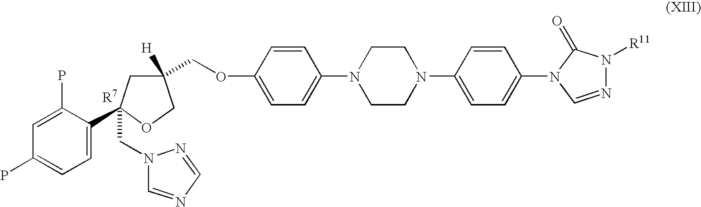
wherein R¹¹ is 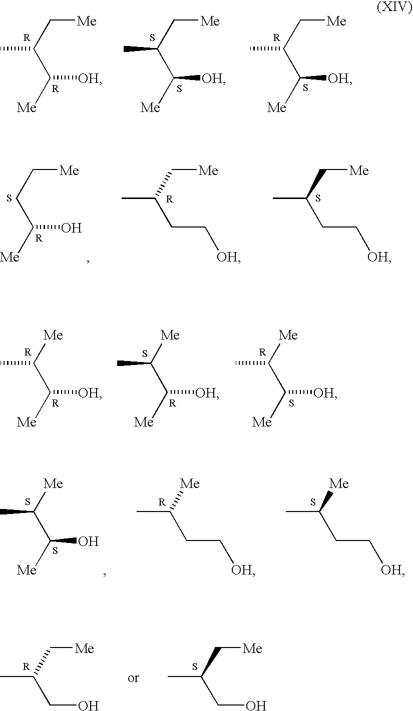
an ester or ether thereof.

In one aspect the ester or ether is a group convertible in vivo into OH e.g. a polyether ester, phosphate ester or an amino acid ester.

In another aspect of the present invention there is provided a compound represent by the formula (XV) 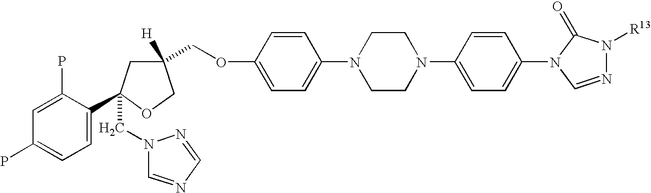
wherein R¹³ is —*CH(C₂H₅)CH(R¹²)CH₃ or —*CH(CH₃)CH(R¹²)CH₃
wherein R¹² is OH, or a group convertible in vivo into OH.

In certain embodiments, the novel soluble crystalline forms of cis-itraconazole, posaconazole or saperconazole have a solubility greater than 5 micrograms/mL, greater than 10 micrograms/mL, greater than 20 micrograms/mL, greater than 30 micrograms/mL, greater than 40 micrograms/mL, greater than 50 micrograms/mL, greater than 100 micrograms/mL, greater than 1 mg/mL, or greater than 10 mg/mL in a solution with a pH of about 1.

Novel soluble crystalline forms of cis-itraconazole, posaconazole or saperconazole of the invention include dicarboxylic acid co-crystals of saperconazole such as saperconazole DL-tartaric acid co-crystal, saperconazole succinic acid co-crystal, saperconazole L-malic acid co-crystal, saperconazole fumaric acid co-crystal, and saperconazole glutaric acid co-crystal. Dicarboxylic acid salts of saperconazole include, but are not limited to, saperconazole mesylate and saperconazole tartrate.

It has surprisingly been found that when an active pharmaceutical ingredient (API) and a selected co-crystal forming compound are allowed to form co-crystals, the resulting co-crystals can give rise to improved properties of the API, as compared to the API in a free form (e.g, free bases, ions, hydrates, solvates, etc.), or an acid salt thereof particularly with respect to: solubility, dissolution, bioavailability, stability, Cₘₐₓ, Tₘₐₓ, processability, longer lasting therapeutic plasma concentration, hygroscopicity, crystallization of amorphous compounds, decrease in form diversity (including polymorphism and crystal habit), change in morphology or crystal habit, etc. For example, a co-crystal form of an API is particularly advantageous where the original API, such as the conazoles including cis-itraconazole, posaconazole or saperconazole is insoluble or sparingly soluble in water. Additionally, the co-crystal properties conferred upon the API are also useful because the bioavailability of the API can be improved and the plasma concentration and/or serum concentration of the API can be improved. This is particularly advantageous for orally-administrable formulations. Moreover, the dose response of the API can be improved, for example by increasing the maximum attainable response and/or increasing the potency of the API by increasing the biological activity per dosing equivalent.

Accordingly, in one aspect, the present invention provides a pharmaceutical composition comprising a co-crystal of a conazole including cis-itraconazole, posaconazole or saperconazole and a co-crystal forming compound, such that the conazole and co-crystal forming compound are capable of co-crystallizing from a solution phase under crystallization conditions or from the solid-state through grinding or heating. In another aspect, the conazole has at least one functional group selected from the group consisting of: ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, sp2 amine, thiocyanate, cyanamide, oxime, nitrile diazo, organohalide, nitro, S-heterocyclic ring, thiophene, N-heterocyclic ring, pyrrole, O-heterocyclic ring, furan, epoxide, peroxide, hydroxamic acid, imidazole, and pyridine and the co-crystal forming compound has at least one functional group selected from the group consisting of: amine, amide, pyridine, imidazole, indole, pyrrolidine, carbonyl, carboxyl, hydroxyl, phenol, sulfone, sulfonyl, mercapto and methyl thio, such that the conazole and co-crystal forming compound are capable of co-crystallizing from a solution phase under crystallization conditions.

Co-crystal formation may be facilitated using one or more of several techniques. Several methods for the formation of co-crystals include, but are not limited to:

a) High Throughput crystallization using the CrystalMax™ platform CrystalMax™ comprises a sequence of automated, integrated high throughput robotic stations capable of rapid generation, identification and characterization of polymorphs, salts, and co-crystals of APIs and API candidates. Worksheet generation and combinatorial mixture design is carried out using proprietary design software Architect™. Typically, an API or an API candidate is dispensed from an organic solvent into tubes and dried under a stream of nitrogen. Salts and/or co-crystal formers may also be dispensed and dried in the same fashion. Water and organic solvents may be combinatorially dispensed into the tubes using a multi-channel dispenser. Each tube in a 96-tube array is then sealed within 15 seconds of combinatorial dispensing to avoid solvent evaporation. The mixtures are then rendered supersaturated by heating to 70 degrees C. for 2 hours followed by a 1 degree C./minute cooling ramp to 5 degrees C. Optical checks are then conducted to detect crystals and/or solid material. Once a solid has been identified in a tube, it is isolated through aspiration and drying. Raman spectra are then obtained on the solids and cluster classification of the spectral patterns is performed using proprietary software (Inquire™).

B) Crystallization from Solution

Co-crystals may be obtained by dissolving the separate components in a solvent and adding one to the other. The co-crystal may then precipitate or crystallize as the solvent mixture is evaporated slowly. The co-crystal may also be obtained by dissolving the two components in the same solvent or a mixture of solvents. The co-crystal may also be obtained by seeding a saturated solution of the two components and seeding with a ground mixture of the co-crystal.

C) Crystallization from the Melt (Co-Melting)

A co-crystal may be obtained by melting the two components together (i.e., co-melting) and allowing recrystallization to occur. In some cases, an anti-solvent may be added to facilitate crystallization.

D) Thermal Microscopy

A co-crystal may be obtained by melting the higher melting component on a glass slide and allowing it to recrystallize. The second component is then melted and is also allowed to recrystallize. The co-crystal may form as a separated phase/band in between the eutectic bands of the two original components.

E) Mixing and/or Grinding

A co-crystal may be obtained by mixing or grinding two components together in the solid state. In one embodiment, a co-crystal is prepared via milling or grinding an API with a co-crystal former. In another embodiment, a co-crystal is prepared via milling or grinding an API, a co-crystal former, and a small amount of solvent. In another embodiment, a co-crystal is prepared with the addition of solvent, without the addition of solvent, or both. Solvents used in such a co-crystallization process can be, for example, but not limited to, acetone, methanol, ethanol, isopropyl alcohol, ethyl acetate, isopropyl acetate, nitromethane, dichloromethane, chloroform, toluene, propylene glycol, dimethyl sulfoxide (DMSO), dimethyl formamide (DMF), diethyl ether (ether), ethyl formate, hexane, acetonitrile, benzyl alcohol, water, or another organic solvent including alcohols.

f) Co-Sublimation

A co-crystal may be obtained by co-subliming a mixture of an API and a co-crystal former in the same sample cell as an intimate mixture either by heating, mixing or placing the mixture under vacuum. A co-crystal may also be obtained by co-sublimation using a Kneudsen apparatus where the API and the co-crystal former are contained in separate sample cells, connected to a single cold finger, each of the sample cells is maintained at the same or different temperatures under a vacuum atmosphere in order to co-sublime the two components onto the cold-finger forming the desired co-crystal.

In each process according to the invention, there is a need to contact the conazole with the co-crystal forming compound. This may involve grinding the two solids together or melting one or both components and allowing them to recrystallize. This may also involve either solubilizing the conazole and adding the co-crystal forming compound, or solubilizing the co-crystal forming compound and adding the conazole. In one embodiment, the conazole may be solubilized in the co-crystal forming compound. Crystallization conditions are applied to the conazole and co-crystal forming compound. This may entail altering a property of the solution, such as pH or temperature and may require concentration of solute, usually by removal of the solvent, typically by drying the solution. Solvent removal results in the concentration of conazole increasing over time so as to facilitate crystallisation. Once the solid phase comprising any crystals is formed, this may be tested as described herein.

The co-crystals obtained as a result of such process steps may be readily incorporated into a pharmaceutical composition by conventional means. Pharmaceutical compositions in general are discussed in further detail below and may further comprise a pharmaceutically-acceptable diluent, excipient, or carrier.

In a further aspect, the present invention provides a process for the production of a pharmaceutical composition, which process comprises:

(1) providing a conazole;

(2) providing a co-crystal forming compound which has at least one functional group selected from the group consisting of: amine, amide, pyridine, imidazole, indole, pyrrolidine, carboxyl, carboxyl, hydroxyl, phenol, sulfone, sulfonyl, mercapto and methyl thio;

(3) grinding, heating, co-subliming, co-melting, or contacting in solution the conazole with the co-crystal forming compound under crystallization conditions, and

(4) isolating co-crystals formed thereby; and

(5) incorporating the co-crystals into a pharmaceutical composition.

In a still further aspect the present invention provides a process for the production of a pharmaceutical composition, which comprises:

(1) grinding, heating, co-subliming, co-melting, or contacting in solution a conazole with a co-crystal forming compound, under crystallization conditions, so as to form a solid phase;

(2) isolating co-crystals comprising the conazole and the co-crystal forming compound.

Assaying the solid phase for the presence of co-crystals of the conazole and the co-crystal forming compound may be carried out by conventional methods known in the art. For example, it is convenient and routine to use powder X-ray diffraction techniques to assess the presence of the co-crystals. This may be affected by comparing the diffractograms of the conazole, the crystal forming compound and putative co-crystals in order to establish whether or not true co-crystals have been formed. Other techniques, used in an analogous fashion, include differential scanning calorimetry (DSC), thermogravimetric analysis (TGA) and Raman spectroscopy. Single crystal X-ray diffraction is especially useful in identifying co-crystal structures.

In a further aspect, the present invention therefore provides a process of screening for co-crystal compounds, which comprises:

(1) providing (i) a conazole compound, and (ii) a co-crystal forming compound;

(2) screening for co-crystals of conazoles with co-crystal forming compounds by subjecting each combination of conazole and co-crystal forming compound to a step comprising:

(a) grinding, heating, co-subliming, co-melting, or contacting in solution the conazole with the co-crystal forming compound under crystallization conditions so as to form a solid phase;

(b) isolating co-crystals comprising the conazole and the co-crystal forming compound.

An alternative embodiment is drawn to a process of screening for co-crystal compounds, which comprises:

(1) providing (i) a conazole or a plurality of different conazoles, and (ii) a co-crystal forming compound or a plurality of different co-crystal forming compounds, wherein at least one of the conazole and the co-crystal forming compound is provided as a plurality thereof;

(2) screening for co-crystals of conazoles with co-crystal forming compounds by subjecting each combination of conazole and co-crystal forming compound to a step comprising

(a) grinding, heating, co-subliming, co-melting, or contacting in solution the conazole with the co-crystal forming compound under crystallization conditions so as to form a solid phase;

(b) isolating co-crystals comprising the conazole and the co-crystal forming compound.

Solubility Modulation:

In a further aspect, the present invention provides a process for modulating the solubility of a conazole, which process comprises:

(1) grinding, heating, co-subliming, co-melting, or contacting in solution the conazole with a co-crystal forming compound under crystallization conditions, so as to form a co-crystal of the conazole and the co-crystal forming compound;

(2) isolating co-crystals comprising the conazole and the co-crystal forming compound.

In one embodiment, the solubility of the conazole is modulated such that the aqueous solubility is increased. Solubility of conazoles may be measured by any conventional means such as spectroscopic determination of the amount of conazole in a saturated solution of the conazole, such as UV-spectroscopy, IR-spectroscopy, Raman spectroscopy, quantitative mass spectroscopy or gas chromatography.

In another aspect of the invention, the conazole may have low aqueous solubility. Typically, low aqueous solubility in the present application refers to a compound having a solubility in water which is less than or equal to 10 mg/mL, when measured at 37° C., or less than or equal to 5 mg/mL or 1 mg/mL. “Low aqueous solubility” can further be specifically defined as less than or equal to 900, 800, 700, 600, 500, 400, 300, 200, 150, 100, 90, 80, 70, 60, 50, 40, 30, 20 micrograms/mL, or further 10, 5 or 1 micrograms/mL. As embodiments of the present invention, solubility can be increased 2, 3, 4, 5, 7, 10, 15, 20, 25, 50, 75, 100, 200, 300, 500, 750, 1000, 5000, or 10,000 times when compared to crystalline free base, by making a co-crystal of the free form or salt. Further aqueous solubility can be measured in simulated gastric fluid (SGF) or simulated intestinal fluid (SIF) rather than water (Dressman J B, et al., Pharm Res. (1998) January; 15(1): 11-22 incorporated by reference in its entirety). SGF (non-diluted) of the present invention is made by combining 1 g/L Triton X-100 and 2 g/L NaCl in water and adjusting the pH with 20 mM HCl to obtain a solution with a final pH=1.7. SIF is 0.68% monobasic potassium phosphate, 1% pancreatin, and sodium hydroxide where the pH of the final solution is 7.5. The pH may also be specified as 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, or 12.0.

Examples of embodiments includes: co-crystal compositions with an aqueous solubility, at 37 degrees C. and a pH of 7.0, that is increased at least 5 fold over the crystalline free form and co-crystal compositions with a solubility in SGF that is increased at least 5 fold over the crystalline free form.

Dissolution Modulation:

In another aspect of the present invention, the dissolution profile of the conazole is modulated whereby the aqueous dissolution rate or the dissolution rate in simulated gastric fluid or in simulated intestinal fluid, or in a solvent or plurality of solvents is increased or decreased. Dissolution rate is the rate at which conazole solids dissolve in a dissolution media. Conazoles that are not dissolved before they are removed from intestinal absorption site are considered useless. Therefore, the rate of dissolution has a major impact on the performance of conazoles that are poorly soluble. Because of this factor, the dissolution rate of conazoles in solid dosage forms is an important, routine, quality control parameter used in the conazole manufacturing process.
Dissolution rate=KS(Cₛ−C)
where K is dissolution rate constant, S is the surface area, Cₛ is the apparent solubility, and C is the concentration of conazole in the dissolution media.

The dissolution rate of conazoles may be measured by conventional means known in the art.

The increase in the dissolution rate of a co-crystal, as compared to the crystalline free form, may be specified, such as by 10, 20, 30, 40, 50, 60, 70, 80, 90, or 100%, or by 2, 3, 4, 5, 6, 7, 8, 9, 10, 15, 20, 25, 30, 40, 50, 75, 100, 125, 150, 175, 200 fold greater than the free form or salt form in the same solution. Conditions under which the dissolution rate is measured is the same as discussed above The increase in dissolution may be further specified by the time the composition remains supersaturated.

Examples of above embodiments includes: co-crystal compositions with an dissolution rate, at 37 degrees C. and a pH of 7.0, that is increased at least 5 fold over the crystalline free form and co-crystal compositions with a dissolution rate in SGF that is increased at least 5 fold over the crystalline free form.

Bioavailability Modulation:

The methods of the present invention are used to make a pharmaceutical conazole formulation with greater solubility, dissolution, and bioavailability, AUC, reduced time to Tₘₐₓ, the time to reach peak blood serum levels, and higher Cₘₐₓ, the maximum blood serum concentration, when compared to the neutral form or salt alone.

AUC is the area under the plot of plasma concentration of conazole (not logarithm of the concentration) against time after conazole administration. The area is conveniently determined by the “trapezoidal rule”: the data points are connected by straight line segments, perpendiculars are erected from the abscissa to each data point, and the sum of the areas of the triangles and trapezoids so constructed is computed. When the last measured concentration (Cₙ, at time tₙ) is not zero, the AUC from tₙ to infinite time is estimated by Cₙ/kₑₗ.

The AUC is of particular use in estimating bioavailability of conazoles, and in estimating total clearance of conazoles (Cl_{T}). Following single intravenous doses, AUC=D/Cl_{T}, for single compartment systems obeying first-order elimination kinetics; alternatively, AUC=C₀/kₑₗ. With routes other than the intravenous, for such systems, AUC=F·D/Cl_{T}, where F is the availability of the conazole.

Thus, in a further aspect, the present invention provides a process for modulating the bioavailability of a conazole when administered in its normal and effective dose range, whereby the AUC is increased, the time to Tₘₐₓ is reduced, or Cₘₐₓ is increased, which process comprises:

(1) grinding, heating, co-subliming, co-melting, or contacting in solution the conazole with a co-crystal forming compound under crystallization conditions, so as to form a co-crystal of the conazole and the co-crystal forming compound;

(2) isolating co-crystals comprising the conazole and the co-crystal forming compound.

Examples of the above embodiments includes: co-crystal compositions with a time to Tₘₐₓ that is reduced by at least 10% as compared to the free crystalline form, co-crystal compositions with a time to Tₘₐₓ that is reduced by at least 20% over the free crystalline form, co-crystal compositions with a time to Tₘₐₓ that is reduced by at least 40% over the free crystalline form, co-crystal compositions with a time to Tₘₐₓ that is reduced by at least 50% over the free crystalline form, co-crystal compositions with a Tₘₐₓ that is reduced by at least 60% over the free crystalline form, co-crystal compositions with a Tₘₐₓ that is reduced by at least 70% over the free crystalline form, co-crystal compositions with a Tₘₐₓ that is reduced by at least 80% over the free crystalline form, co-crystal compositions with a Cₘₐₓ that is increased by at least 20% over the free crystalline form, co-crystal compositions with a Cₘₐₓ that is increased by at least 30% over the free crystalline form, co-crystal compositions with a Cₘₐₓ that is increased by at least 40% over the free crystalline form, co-crystal compositions with a Cₘₐₓ that is increased by at least 50% over the free crystalline form, co-crystal compositions with a Cₘₐₓ that is increased by at least 60% over the free crystalline form, co-crystal compositions with a Cₘₐₓ that is increased by at least 70% over the free crystalline form, co-crystal compositions with a Cₘₐₓ that is increased by at least 80% over the free crystalline form, co-crystal compositions with an AUC that is increased by at least 10% over the free crystalline form, co-crystal compositions with an AUC that is increased by at least 20% over the free crystalline form, co-crystal compositions with an AUC that is increased by at least 30% over the free crystalline form, co-crystal compositions with an AUC that is increased by at least 40% over the free crystalline form, co-crystal compositions with an AUC that is increased by at least 50% over the free crystalline form, co-crystal compositions with an AUC that is increased by at least 60% over the free crystalline form, co-crystal compositions with an AUC that is increased by at least 70% over the free crystalline form, or co-crystal compositions with an AUC that is increased by at least 80% over the free crystalline form.

Dose Response Modulation:

In a further aspect the present invention provides a process for improving the dose response of a conazole, which process comprises:

(1) grinding, heating, co-subliming, co-melting, or contacting in solution a conazole with a co-crystal forming compound under crystallization conditions, so as to form a co-crystal of the conazole and the co-crystal forming compound;

(2) isolating co-crystals comprising the conazole and the co-crystal forming compound.

Dose response is the quantitative relationship between the magnitude of response and the dose inducing the response and may be measured by conventional means known in the art. The curve relating effect (as the dependent variable) to dose (as the independent variable) for a conazole-cell system is the “dose-response curve”. Typically, the dose-response curve is the measured response to a conazole plotted against the dose of the conazole (mg/kg) given. The dose response curve can also be a curve of AUC against the dose of the conazole given.

Increased Stability:

In a still further aspect the present invention provides a process for improving the stability of a conazole in its free form or a salt thereof, which process comprises:

(1) Grinding, heating, co-subliming, co-melting, or contacting in solution the pharmaceutical salt with a co-crystal forming compound under crystallization conditions, so as to form a co-crystal of the conazole and the co-crystal forming compound;

(2) isolating co-crystals comprising the conazole and the co-crystal forming compound.

In another embodiment, the compositions of the present invention, including the conazole or active pharmaceutical ingredient (conazole) and formulations comprising the conazole, are suitably stable for pharmaceutical use. For example, the conazole or formulations thereof of the present invention are stable such that when stored at 30 deg. C. for 2 years, less than 0.2% of any one degradant is formed. The term degradant refers herein to product(s) of a single type of chemical reaction. For example, if a hydrolysis event occurs that cleaves a molecule into two products, for the purpose of the present invention, it would be considered a single degradant. In another embodiment, when stored at 40 deg. C. for 2 years, less than 0.2% of any one degradant is formed. Alternatively, when stored at 30 deg. C. for 3 months, less than 0.2% or 0.15%, or 0.1% of any one degradant is formed, or when stored at 40 deg. C. for 3 months, less than 0.2% or 0.15%, or 0.1% of any one degradant is formed. Further alternatively, when stored at 60 deg. C. for 4 weeks, less than 0.2% or 0.15%, or 0.1% of any one degradant is formed. The relative humidity (RH) may be specified as ambient (RH), 75% (RH), or as any single integer between 1 to 99%.

Difficult to Salt or Unsaltable Compounds:

In a still further aspect the present invention provides a process for making co-crystals of unsaltable conazoles which process comprises

(1) Grinding, heating, co-subliming, co-melting, or contacting in solution a conazole with a co-crystal forming compound under crystallization conditions, so as to form a co-crystal of the conazole and the co-crystal forming compound;

(2) isolating co-crystals comprising the conazole and the co-crystal forming compound.

Difficult to salt compounds include bases with a pKa<3 or acids with a pKa>10. Zwitterions are also difficult to salt or unsaltable compounds.

Decreasing Hygroscopicity:

In a still further aspect the present invention provides a method for decreasing the hygroscopicity of a conazole, which method comprises

(1) Grinding, heating, co-subliming, co-melting, or contacting in solution the conazole with a co-crystal forming compound under crystallization conditions;

(3) forming a co-crystal of the conazole and the co-crystal forming compound;

(2) isolating co-crystals comprising the conazole and the co-crystal forming compound.

An aspect of the present invention provides a pharmaceutical composition comprising a co-crystal of a conazole that is less hygroscopic than amorphous or crystalline, free form or salt (including metal salts such as sodium, potassium, lithium, calcium, magnesium). Hygroscopicity can be assessed by dynamic vapor sorption analysis, in which 5-50 mg of the compound is suspended from a Cahn microbalance. The compound being analyzed should be placed in a non-hygroscopic pan and its weight should be measured relative to an empty pan composed of identical material and having nearly identical size, shape, and weight. Ideally, platinum pans should be used. The pans should be suspended in a chamber through which a gas, such as air or nitrogen, having a controlled and known percent relative humidity (% RH) is flowed until eqilibrium criteria are met. Typical equilibrium criteria include weight changes of less than 0.01% change over 3 minutes at constant humidity and temperature. The relative humidity should be measured for samples dried under dry nitrogen to constant weight (<0.01% change in 3 minutes) at 40 degrees C. unless doing so would de-solvate or otherwise convert the material to an amorphous compound. In one aspect, the hygroscopicity of a dried compound can be assessed by increasing the RH from 5 to 95% in increments of 5% RH and then decreasing the RH from 95 to 5% in 5% increments to generate a moisture sorption isotherm. The sample weight should be allowed to equilibrate between each change in % RH. If the compound deliquesces or becomes amorphous between above 75% RH, but below 95% RH, the experiment should be repeated with a fresh sample and the relative humidity range for the cycling should be narrowed to 5-75% RH or 10-75% RH instead of 5-95% RH. If the sample cannot be dried prior to testing due to lack of form stability, than the sample should be studied using two complete humidity cycles of either 10-75% RH or 5-95% RH, and the results of the second cycle should be used if there is significant weight loss at the end of the first cycle.

Hygroscopicity can be defined using various parameters. For purposes of the present invention, a non-hygroscopic molecule should not gain or lose more than about 1.0%, or about 0.5% weight at 25 degrees C. when cycled between 10 and 75% RH (relative humidity at 25 degrees C.). The non-hygroscopic molecule, in one embodiment, should not gain or lose more than 1.0%, or about 0.5% weight when cycled between 5 and 95% RH at 25 degrees C., or more than 0.25% of its weight between 10 and 75% RH. In another embodiment, a non-hygroscopic molecule will not gain or lose more than 0.25% of its weight when cycled between 5 and 95% RH.

Alternatively, for purposes of the present invention, hygroscopicity can be defined using the parameters of Callaghan et al., Equilibrium moisture content of pharmaceutical excipients, in Conazole Dev. Ind. Pharm., Vol. 8, pp. 335-369 (1982). Callaghan et al. classified the degree of hygroscopicity into four classes.

| | |
|---|---|
| Class 1: | Essentially no moisture increases occur at |
| Non-hygroscopic | relative humidities below 90%. |
| Class 2: | Essentially no moisture increases occur at |
| Slightly hygroscopic | relative humidities below 80%. |
| Class 3: | Moisture content does not increase more |
| Moderately hygroscopic | than 5% after storage for 1 week at |
| | relative humidities below 60%. |
| Class 4: | Moisture content increase may occur at |
| Very hygroscopic | relative humidities as low as 40 to 50%. |

Alternatively, for purposes of the present invention, hygroscopicity can be defined using the parameters of the European Pharmacopoeia Technical Guide (1999, p. 86) which has defined hygrospocity, based on the static method, after storage at 25° C. for 24 h at 80 percent RH:

Slightly hygroscopic: Increase in mass is less than 2 percent m/m and equal to or greater than 0.2 percent m/m.

Hygroscopic: Increase in mass is less than 15 percent m/m and equal to or greater than 0.2 percent m/m.

Very Hygroscopic: Increase in mass is equal to or greater than 15 percent m/m.

Deliquescent: Sufficient water is absorbed to form a liquid.

Co-crystals of the present invention can be set forth as being in Class 1, Class 2, or Class 3, or as being Slightly hygroscopic, Hygroscopic, or Very Hygroscopic. Co-crystals of the present invention can also be set forth based on their ability to reduce hygroscopicity. Thus, several co-crystals of the present invention are less hygroscopic than the conazole. The reference compound can be specified as the conazole in free form (free acid, free base, hydrate, solvate, etc.) or salt (e.g., metal salt such as sodium, potassium, lithium, calcium, or magnesium). Further included in the present invention are co-crystals that do not gain or lose more than 1.0% weight at 25 degrees C. when cycled between 10 and 75% RH, wherein the reference compound gains or loses more than 1.0% weight under the same conditions. Further included in the present invention are co-crystals that do not gain or lose more than 0.5% weight at 25 degrees C. when cycled between 10 and 75% RH, wherein the reference compound gains or loses more than 0.5% or more than 1.0% weight under the same conditions. Further included in the present invention are co-crystals that do not gain or lose more than 1.0% weight at 25 degrees C. when cycled between 5 and 95% RH, wherein the reference compound gains or loses more than 1.0% weight under the same conditions. Further included in the present invention are co-crystals that do not gain or lose more than 0.5% weight at 25 degrees C. when cycled between 5 and 95% RH, wherein the reference compound gains or loses more than 0.5% or more than 1.0% weight under the same conditions. Further included in the present invention are co-crystals that do not gain or lose more than 0.25% weight at 25 degrees C. when cycled between 5 and 95% RH, wherein the reference compound gains or loses more than 0.5% or more than 1.0% weight under the same conditions.

Further included in the present invention are co-crystals that have a hygroscopicity (according to Callaghan et al.) that is at least one class lower than the reference compound or at least two classes lower than the reference compound. Included are a Class 1 co-crystals of a Class 2 reference compound, a Class 2 co-crystals of a Class 3 reference compound, a Class 3 co-crystals of a Class 4 reference compound, a Class 1 co-crystals of a Class 3 reference compound, a Class 1 co-crystals of a Class 4 reference compound, or a Class 2 co-crystals of a Class 4 reference compound.

Further included in the present invention are co-crystals that have a hygroscopicity (according to the European Pharmacopoeia Technical Guide) that is at least one class lower than the reference compound or at least two classes lower than the reference compound. Non-limiting examples include; a slightly hygroscopic co-crystals of a hygroscopic reference compound, a hygroscopic co-crystals of a very hygroscopic reference compound, a very hygroscopic co-crystals of a deliquescent reference compound, a slightly hygroscopic co-crystals of a very hygroscopic reference compound, a slightly hygroscopic co-crystals of a deliquescent reference compound, and a hygroscopic co-crystals of a deliquescent reference compound.

In one aspect, the present invention demonstrates that crystalline phases can be engineered by combining molecules selected to match hydrogen bond donors with acceptors and by considering structural complementarities. The present invention further shows that supramolecular synthesis can be applied to active pharmaceutical ingredients using organic acid and base combinations with pKₐ differences that are inconsistent with salt formation in water (given the pKₐ value of 3.7 for the piperazine of itraconazole, conventional wisdom would limit a salt screen to those strong acids having dissociation constants below 1.7).

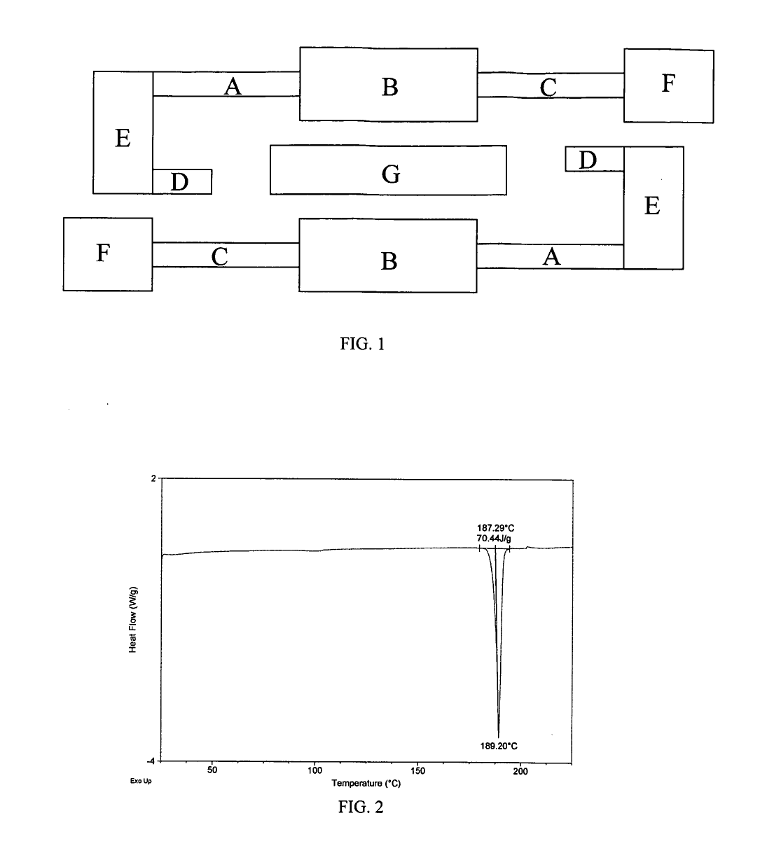

An aspect of the present invention includes co-crystals comprising or consisting of hydrogen-bonded trimers consisting of two molecules of cis-itraconazole (or two molecules of posaconazole or two molecules of saperconazole) and one molecule of a dicarboxylic acid (e.g., succinic acid). Several dicarboxylic acid co-crystals of cis-itraconazole, posaconazole or saperconazole have a crystal structure as shown in FIG. 1. The drug molecule (see FIG. 1) is composed of a three ring backbone (A-C), a triazole ring (D), a spacer group (E) and a terminating group (F). The trimer has two drug molecules oriented anti-parallel to each other with a second molecule, a dicarboxylic acid, (G) templating or filling the void between the two drug molecules. The distance between the carboxylic acid oxygen (—O(H)), one of the possible function groups of molecule G, and the triazole nitrogen (—N—), D, can be between 3.4 and 1.8 angstroms, between 3.2 and 2.3 angstroms, between 3.0 and 2.5 angstroms, or between 2.8 and 2.6 angstroms. The distance between the two drug molecules that make up the trimer, as measured by the distance between a nitrogen atom in ring A of one molecule and ring A of the second molecule can be between about 7.5 and about 6.4 angstroms, between about 7.0 and about 6.6 angstroms, or about 6.8 angstroms. The distance between the two triazole rings (D) in the trimer, as measured by the shortest distance between two nitrogen atoms, with one each separate molecule, can be between about 12.5 and about 8.0 angstroms, between about 11 and about 10.6 angstroms, or about 10.8 angstroms. The trimer can also, in some cases, be defined further by being oriented around a center of inversion located at the center of molecule G. The dicarboxylic acid that is used to fill the pocket of the trimer can be for example, but not limited to, fumaric acid, succinic acid, tartaric acid, DL-tartaric acid, D-tartaric acid, L-tartaric acid, meso-tartaric acid, D-malic acid, L-malic acid, DL-malic acid, malonic acid, glutaric acid, adipic acid or acetic acid. The crystal structure of one congener reveals an unanticipated and specific interaction between the triazole of the conazole and the diacid in the solid state (See, for example, U.S. patent application Ser. No. 10/449,307, filed on May 30, 2003, which is hereby incorporated by reference in its entirety).

A further embodiment of the invention encompasses a method of treating or preventing local or systemic fungal, yeast, and dermatophyte infections in a patient which comprises administering to a patient in need of such treatment or prevention a therapeutically or prophylactically effective amount of a pharmaceutically acceptable soluble crystalline form of a conazole (including cis-itraconazole, posaconazole or saperconazole), including salts, co-crystals, salt co-crystals and hydrates, solvates or polymorphs thereof. More specifically, the invention includes a method for treating or preventing local and systemic fungal, yeast, and dermatophyte infections in a patient comprising administering to a patient in need of such treatment or prevention, a therapeutically or prophylactically effective amount of a composition of the present invention including a salt or co-crystal of saperconazole such as saperconazole DL-tartaric acid co-crystal, saperconazole fumaric acid co-crystal, saperconazole succinic acid co-crystal, saperconazole L-malic acid co-crystal, saperconazole glutaric acid co-crystal, or saperconazole mesylate salt.

The invention further encompasses the use of a dicarboxylic acid salt or co-crystal of cis-itraconazole, posaconazole or saperconazole. Methods of treatment include administration of pharmaceutical compositions of the invention comprising a therapeutically effective amount of an acid salt of cis-itraconazole, posaconazole or saperconazole, or a soluble, multicomponent crystalline system comprising cis-itraconazole, posaconazole or saperconazole, or a co-crystal comprising cis-itraconazole, posaconazole or saperconazole.

Pharmaceutical Compositions and Dosage Forms

Pharmaceutical dosage forms of the invention comprise a therapeutically or prophylactically effective amount of a novel soluble crystalline form of cis-itraconazole, posaconazole or saperconazole, including hydrates, solvates or polymorphs thereof. These dosage forms also comprise a soluble, multicomponent crystalline system comprising cis-itraconazole, posaconazole or saperconazole organic salt and an organic solvent. These compositions can be administered orally, parenterally, by inhalation spray, topically, rectally, nasally, buccally, vaginally or via an implanted reservoir. Oral pharmaceutical compositions and dosage forms are dosage forms in another embodiment. For example, the oral dosage form can be a solid dosage form, such as a tablet, a caplet, a hard gelatin capsule, a starch capsule, a hydroxypropyl methylcellulose (HPMC) capsule, or a soft elastic gelatin capsule. Other dosage forms include an intradermal dosage form, an intramuscular dosage form, a subcutaneous dosage form, and an intravenous dosage form.

Pharmaceutical compositions and dosage forms of the invention comprise an active ingredient as disclosed herein, e.g., an acid salt or a co-crystal of cis-itraconazole, posaconazole or saperconazole or a soluble, multicomponent crystalline system comprising cis-itraconazole, posaconazole or saperconazole organic salt and an organic solvent. Pharmaceutical compositions and unit dosage forms of the invention typically also comprise one or more pharmaceutically acceptable excipients or diluents. In one embodiment, the pharmaceutical compositions and unit dosage forms of the invention typically also comprise one or more pharmaceutically acceptable excipients or diluents, wherein at least one of the pharmaceutically acceptable excipients or diluents is an antioxidant.

Pharmaceutical unit dosage forms of this invention are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules, starch capsules, hydroxypropyl methylcellulose (HPMC) capsules, and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease or disorder may contain larger amounts of the active ingredient than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990) or Remington: The Science and Practice of Pharmacy, 19th ed., Mack Publishing, Easton Pa. (1995).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. In addition, pharmaceutical compositions or dosage forms may contain one or more compounds that reduce or alter the rate by which the active ingredient will decompose. Such compounds, which are referred to herein as “stabilizers”, include, but are not limited to, antioxidants, pH buffers, or salt buffers.

One or more antioxidants can be used in pharmaceutical compositions and dosage forms to deter radical oxidation of the active ingredient, wherein such antioxidants include, but are not limited to, ascorbic acid, phenolic antioxidants including, but not limited to, butylated hydroxyanisole (BHA) and propyl gallate, and chelators including, but not limited to citrate, EDTA, and DTPA. In cases where radical oxidation of the active ingredient is known to occur, a combination of phenolic antioxidants and chelators can be used.

Like the amounts and types of excipients, the amounts and specific type of active ingredient in a dosage form may differ depending on factors such as, but not limited to, the route by which it is to be administered to patients. However, typical dosage forms of the invention comprise a pharmaceutically acceptable salt or co-crystal of cis-itraconazole, posaconazole or saperconazole or its stereoisomers, and pharmaceutically acceptable hydrates, solvates, polymorphs, and co-crystals thereof, in an amount of from about 10 mg to about 1000 mg, from about 25 mg to about 500 mg, from about 40 mg to about 400 mg, or from about 50 mg to about 200 mg.

According to the present invention, the packaging of pharmaceutical compositions can be accomplished via a container for containing the pharmaceutical compositions and may also include divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

A non-limiting example of such a container is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of an optionally transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. In one embodiment, the strength of the sheet is such that the tablets or capsules can be removed from the blister pack by manually applying pressure on the recesses whereby an opening is formed in the sheet at the place of the recess. The tablet or capsule can then be removed via said opening.

Oral Dosage Forms

Pharmaceutical compositions of the invention that are suitable for oral administration can be presented as discrete dosage forms, such as, but not limited to, tablets (including without limitation scored or coated tablets), pills, caplets, capsules (including without limitation hard gelatin capsules, starch capsules, HPMC capsules, and soft elastic gelatin capsules), chewable tablets, powder packets, sachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the active ingredient, and may be prepared by methods of pharmacy well known to those skilled in the art. See generally, Remington's Pharmaceutical Sciences, 18th ed., Mack Publishing, Easton Pa. (1990) or Remington: The Science and Practice of Pharmacy, 19th ed., Mack Publishing, Easton Pa. (1995).

Typical oral dosage forms of the invention are prepared by combining the active ingredient in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, stabilizers, and disintegrating agents.

Due to their ease of administration, tablets, caplets, and capsules (such as hard gelatin, HPMC, or starch capsules) represent the most advantageous solid oral dosage unit forms, in which case solid pharmaceutical excipients are used. If desired, tablets or caplets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the invention include, but are not limited to, binders, stabilizers, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103, AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants can be used in the pharmaceutical compositions and dosage forms to provide tablets or caplets that disintegrate when exposed to an aqueous environment. Tablets or caplets that contain too much disintegrant may disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the invention. The amount of disintegrant used varies based upon the type of formulation and mode of administration, and is readily discernible to those of ordinary skill in the art. Typical pharmaceutical compositions comprise from about 0.5 to about 15 weight percent of disintegrant, for example, about 1 to about 5 weight percent of disintegrant.

Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other aligns, other celluloses, gums, and mixtures thereof.

Antioxidants can be used in the pharmaceutical compositions and dosage forms to deter degradation or radical oxidation of the active ingredient. Examples of suitable antioxidants include, but are not limited to, ascorbic acid, phenolic antioxidants including, but not limited to, butylated hydroxyanisole (BHA) and propyl gallate, and chelators including, but not limited to, citrate, EDTA, and DTPA, or combinations thereof.

Lubricants that can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Other oral dosage forms for pharmaceutical compositions of the invention are soft elastic gelatin capsules. Soft elastic gelatin capsule unit dosage forms can be made using conventional methods well known in the art. See, e.g., Ebert, Pharm. Tech, 1(5):44-50 (1977). In general, soft elastic gelatin capsules (also known as “soft gels”) have an elastic or soft, globular or oval shaped gelatin shell that is typically a bit thicker than that of hard gelatin capsules, wherein a plasticizing agent, e.g., glycerin, sorbitol, or a similar polyol, is added to a gelatin. The type of gelatin, as well as the amounts of plasticizer and water, can be used to vary the hardness of the capsule shell. The soft gelatin shells may contain a preservative, such as metliyl- and propylparabens and sorbic acid, to prevent the growth of fungi. The active ingredient may be dissolved or suspended in a liquid vehicle or carrier, such as vegetable or mineral oils, glycols, such as polyethylene glycol and propylene glycol, triglycerides, surfactants, such as polysorbates, or a combination thereof.

Delayed Release Dosage Forms

Pharmaceutically acceptable salts and co-crystals of con azoles can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the conazole salts and co-crystals of the invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of conazoles and thus effect controlled delivery of the drug. Examples of specific anion exchangers include, but are not limited to, Duolite® A568 and Duolite® AP143 (Rohm & Haas, Spring House, Pa. USA).

One embodiment of the invention encompasses a unit dosage form which comprises a pharmaceutically acceptable salt or co-crystal of a conazole, or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the invention. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the invention include, but are not limited to, the OROS® Push-Pull™, Delayed Push-Pull™, Multi-Layer Push-Pull™, and Push-Stick™ Systems, all of which are well known. See, e.g., http://www.alza.com. Additional OROS® systems that can be used for the controlled oral delivery of compounds and compositions of the invention include OROS®-CT and L-OROS®. Id.; see also, Delivery Times, vol. II, issue II (Alza Corporation).

Conventional OROS® oral dosage forms are made by compressing a drug powder (e.g., conazole salt) into a hard tablet, coating the tablet with cellulose derivatives to form a semi-permeable membrane, and then drilling an orifice in the coating (e.g., with a laser). (Kim, Cherng-ju, Controlled Release Dosage Form Design, 231-238 Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively deliver drugs with low water solubility. Because conazole salts and complexes of this invention are far more soluble in water than the free base itself, they are well suited for osmotic-based delivery to patients. This invention does, however, encompass the incorporation of the free base, and non-salt isomers and isomeric mixtures thereof, into OROS® dosage forms.

A specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer comprises a salt or a co-crystal of a conazole, or a polymorph, solvate, hydrate, dehydrate, co-crystal, anhydrous, or amorphous form thereof. See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt or co-crystal of a conazole, or a polymorph, solvate, hydrate, dehydrate, co-crystal, anhydrous, or amorphous form thereof. See U.S. Pat. No. 6,342,249, the entirety of which is incorporated herein by reference.

Another example of a delayed-release dosage form that also functions as a time controlled-release dosage form is described in U.S. Pat. No. 5,366,738, herein incorporated by reference in its entirety. The controlled-release drug delivery device described in U.S. Pat. No. 5,366,738 is known as a gel extrusion module (GEM) delivery device. The GEM device is a drug delivery device for the controlled in situ production and release of a dispersion containing a beneficial agent such as a pharmaceutical drug comprising:

(A) a compressed core prepared from an admixture comprising:

(i) a therapeutically effective amount of the beneficial agent; and

(ii) a polymer which upon hydration forms gelatinous microscopic particles; and

(B) a water insoluble, water impermeable polymeric coating comprising a polymer and a plasticizer, which surrounds and adheres to the core, the coating having a plurality of formed apertures exposing between about 1 and about 75% of the core surface; and wherein the release rate of the beneficial agent from the device is a function of the number and size of the apertures.

In the GEM device, the polymer inside the compressed core is selected from materials such as sodium polyacrylate, carboxypolymethylenes and the pharmaceutically acceptable salts thereof such as a sodium salt, wherein the carboxypolymethylenes are prepared from acrylic acid crosslinked with allylethers of sucrose or pentaerythritol, and, for example, it is selected from carboxypolymethylenes prepared from acrylic acid crosslinked with allylethers of sucrose or pentaerythritol, and the pharmaceutically acceptable salts thereof. Often CARBOPOL® 974P and pharmaceutically acceptable salts thereof, particularly the sodium salt, is used as the polymer inside the compressed core. In addition, the compressed core may also contain one or more polymer hydration modulating agents, anti-oxidants, lubricants, fillers and excipients. An optional subcoating may be applied to the compressed core prior to application of the water insoluble coating as an aid in the manufacturing process. The subcoating may be comprised of, for example, hydroxypropyl cellulose and hydroxypropylmethylcellulose. Additional coatings may be applied for aesthetic or functional purposes.

The water insoluble, water impermeable polymeric coating is comprised of, for example, (1) a polymer selected from polyvinyl chloride, cellulose acetate, cellulose acetate butyrate, ethylcellulose and combinations of these polymers; and (2) a plasticizer selected from diethylphthalate, dibutylsebacate and triethylcitrate. For example, the polymeric coating is comprised of cellulose acetate butyrate and triethyl citrate. The GEM device does not function as an osmotic drug delivery device, hence the release function of the device depends on passage of fluids from the external environment of the body to the internal environment of the compressed core through the formed apertures. It is intended that the terms “water insoluble, water impermeable” used to describe the polymeric coating define a coating which is essentially water insoluble and water impermeable, meaning that the polymeric coating allows minimal to no passage of water through the coating from the external environment of the body to the internal environment of the compressed core, except for the fluid passage that occurs through the drilled apertures, during the period of time the drug is being released from the GEM device in the body. Any minimal amount of water that does pass through the water insoluble, water impermeable polymeric coating is insubstantial and does not significantly contribute to the function of the GEM device, i.e. the release rate of the drug through the apertures. Rather the release rate of tizanidine from the GEM device is primarily a function of the number and size of the apertures on the device.

For an elegant, aesthetically pleasing final product, an outer finish coat may finally be applied to the GEM delivery device containing colorants, waxes, and the like. The GEM device can also be enterically coated, either before or after the application of additional finish coatings. Even without enteric coating, extrusion of the polymer which carries tizanidine out from inside the compressed core of the GEM device does not occur to a substantial extent in the acidic pH of the stomach, therefore substantial release of tizanidine should not occur in the stomach. Further details and examples of the GEM delivery device are described in U.S. Pat. No. 5,366,738.

Topical Dosage Forms

Topical dosage forms of the invention include, but are not limited to, creams, lotions, ointments, gels, shampoos, sprays, aerosols, solutions, emulsions, and other forms know to one of skill in the art. See, e.g., Remington's Pharmaceutical Sciences, 18^{th} ed., Mack Publishing, Easton, Pa. (1990); and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity, for example, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, optionally in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18^{th} ed., Mack Publishing, Easton, Pa. (1990).

Parenteral Dosage Forms

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are optionally sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, without limitation: sterile water; Water for Injection USP; saline solution; glucose solution; aqueous vehicles such as but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. The solutions are, optionally, isotonic and have a physiological pH.

Compounds that increase the solubility the active ingredient(s) disclosed herein can also be incorporated into the parenteral dosage forms of the invention.

Transdermal and Mucosal Dosage Forms

Transdermal and mucosal dosage forms of the invention include, but are not limited to, ophthalmic solutions, patches, sprays, aerosols, creams, lotions, suppositories, ointments, gels, solutions, emulsions, suspensions, or other forms know to one of skill in the art. See, e.g., Remington's Pharmaceutical Sciences, 18^{th} ed., Mack Publishing, Easton, Pa. (1990); and Introduction to Pharmaceutical Dosage Forms, 4^{th} ed., Lea & Febiger, Philadelphia, Pa. (1985). Dosage forms suitable for treating mucosal tissues within the oral cavity can be formulated as mouthwashes, as oral gels, or as buccal patches. Further, transdermal dosage forms include “reservoir type” or “matrix type” patches, which can be applied to the skin and worn for a specific period of time to permit the penetration of a desired amount of active ingredient.

Suitable excipients (e.g., carriers and diluents) and other materials that can be used to provide transdermal and mucosal dosage forms encompassed by this invention are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with active ingredients of the invention. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, solvates, polymorphs, or co-crystals of the active ingredient can be used to further adjust the properties of the resulting composition.

Methods of Treatment and Prevention

Pharmaceutically acceptable salts and co-crystals of cis-itraconazole, posaconazole or saperconazole, and pharmaceutical compositions and dosage forms thereof, possess potent activity against and are useful for treating or preventing local and systemic fungal, yeast, and dermatophyte infections. For example, pharmaceutically acceptable soluble crystalline form of cis-itraconazole, posaconazole or saperconazole, and pharmaceutical compositions and dosage forms thereof, can be used to treat or prevent blastomycosis, aspergillosis, histoplasmosis, onychomycosis, coccidioidomycosis, paracoccidioidomycosis, cryptococcosis, dermatophyte, and candidiasis infections.

The magnitude of a prophylactic or therapeutic dose of each active ingredient in the acute or chronic management of a disease or disorder will vary with the disease or disorder itself, the specific active ingredients, and the route of administration. The dose, dose frequency, or both, may also vary according to age, body weight, response, the past medical history of the patient, and consideration of whether the patient is or will be concurrently or concomitantly taking other drugs or pharmaceuticals. Suitable dosing regimens can be readily selected by the skilled artisan with due consideration of such factors by following, for example, dosages and dose regimens reported in the literature and recommended in the Physician's Desk Reference® (56th ed., 2002) for itraconazole or saperconazole; which can be extended for determining dosing of posaconazole. Unless otherwise indicated, the magnitude of a prophylactic or therapeutic dose of the active ingredient used in an embodiment of the invention will be that which is safe and effective (e.g., has received regulatory approval).

In one embodiment of the invention, the active ingredient is administered orally as needed in an amount of from about 10 mg to about 1000 mg, from about 25 mg to about 500 mg, from about 40 mg to about 400 mg, or from about 50 mg to about 200 mg. The dosage amounts can be administered in single or divided doses. The dosage amounts and frequencies provided above are encompassed by the terms “therapeutically effective”, “prophylactically effective”, and “therapeutically or prophylactically effective” as used herein.

The suitability of a particular route of administration employed for a particular active ingredient will depend on the active ingredient itself (e.g., whether it can be administered orally without decomposing prior to entering the blood stream) and the disease or disorder to be treated or prevented. For example, topical administration is optionally used for treating or preventing local diseases or disorders of the skin, while oral or parenteral administration is optionally used for systemic diseases or disorders, or diseases or disorders within the body of the patient. Similarly, oral or parenteral administration may be used for the treatment or prevention of acute diseases or disorders, whereas transdermal or subcutaneous routes of administration may be employed for treatment or prevention of a chronic disease or disorder.

Preparation of Soluble Crystalline Forms of Cis-Itraconazole, Posaconazole or Saperconazole

Soluble crystalline form of cis-itraconazole, posaconazole or saperconazole can be made using various methods known to those skilled in the art. For example, methods for the chemical synthesis of (±)cis-itraconazole are described in U.S. Pat. No. 4,267,179 and Heeres, J. et al., J. Med. Chem., 27:894-900 (1984), both of which are incorporated by reference herein in their entireties. The four individual stereoisomeric forms of the compounds of formula (I), or diastereomeric pairs or mixtures thereof, can be prepared and purified using various methods known to those skilled in the art, such as those described in U.S. Pat. No. 5,998,413 and U.S. Pat. No. 5,474,997, both of which are specifically incorporated herein by reference in their entireties.

Salts and co-crystals of cis-itraconazole, posaconazole or saperconazole, include without limitation, pharmaceutically acceptable salts prepared by treating cis-itraconazole, posaconazole or saperconazole free base with appropriate acids, such as organic or inorganic acids, including without limitation, malic acid, hydrochloric acid, sulfuric acid, fumaric acid, phosphoric acid, tartaric acid, maleic acid, malonic acid, adipic acid, benzenesulfonic acid, and the like. For example, the process for forming a salt or co-crystal can be carried out in a solvent system in which both reactants (i.e., a conazole such as cis-itraconazole, posaconazole or saperconazole free base and the respective acid) are sufficiently soluble. In one method, in order to achieve crystallization or precipitation, a solvent or solvent mixture in which the resulting salt and co-crystal is only slightly soluble or not soluble at all is used. Alternatively, a solvent in which the desired salt and co-crystal is very soluble can be used, and then an anti-solvent (or a solvent in which the resulting salt is poorly soluble) is added to the solution. Other variants for salt formation or crystallization include concentrating the salt and co-crystal solution (e.g., by heating, under reduced pressure if necessary, or by slowly evaporating the solvent, for example, at room temperature), or seeding with the addition of seed crystals, or setting up water activity required for hydrate formation. In another method, cis-itraconazole, posaconazole or saperconazole and a dicarboxylic acid (e.g., a dicarboxylic acid of formula (IV)) are dissolved in a solvent at greater than 60° C., cooled to below room temperature and seeded with a cis-itraconazole, posaconazole or saperconazole salt or co-crystal. Specific examples of the preparation of cis-itraconazole salts and co-crystals can be found below.

The invention is further defined by reference to the following examples. It will be apparent to those skilled in the art that many modifications, both to materials and methods, can be practiced without departing from the scope of this invention.

### EXEMPLIFICATION

Analytical Methods

Differential scanning calorimetric (DSC) analysis of the samples was performed using a Q1000 Differential Scanning Calorimeter (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/98/2000/NT, version 3.1E; Build 3.1.0.40 (2001 TA Instruments-Water LLC).

For the DSC analysis, the purge gas used was dry nitrogen, the reference material was an empty aluminum pan that was crimped, and the sample purge was 50 mL/minute.

DSC analysis of the sample was performed by placing the sample in an aluminum pan with a crimped pan closure. The starting temperature was typically 20 degrees C. with a heating rate of 10 degrees C./minute, and the ending temperature was 200 degrees C. All reported DSC transitions represent the temperature of endothermic or exothermic transition at their respective peaks with an error of +/−2 degrees C., unless otherwise indicated.

Thermogravimetric analysis (TGA) of samples was performed using a Q500 Thermogravimetric Analyzer (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/98/2000/NT, version 3.1E; Build 3.1.0.40 (2001 TA Instruments-Water LLC).

For the TGA experiments, the purge gas used was dry nitrogen, the balance purge was 40 mL/minute N₂, and the sample purge was 60 mL/minute N₂.

TGA was performed on the sample by placing the sample in a platinum pan. The starting temperature was typically 20 degrees C. with a heating rate of 10 degrees C./minute, and the ending temperature was 300 degrees C.

A powder X-ray diffraction (PXRD) pattern for the samples was obtained using a D/Max Rapid, Contact (Rigaku/MSC, The Woodlands, Tex., U.S.A.), which uses as its control software RINT Rapid Control Software, Rigaku Rapid/XRD, version 1.0.0 (1999 Rigaku Co.). In addition, the analysis software used were RINT Rapid display software, version 1.18 (Rigaku/MSC), and JADE XRD Pattern Processing, versions 5.0 and 6.0 ((1995-2002, Materials Data, Inc.).

For the PXRD analysis, the acquisition parameters were as follows: source was Cu with a K line at 1.5406 Å; x-y stage was manual; collimator size was 0.3 mm; capillary tube (Charles Supper Company, Natick, Mass., U.S.A.) was 0.3 mm ID; reflection mode was used; the power to the X-ray tube was 46 kV; the current to the X-ray tube was 40 mA; the omega-axis was oscillating in a range of 0-5 degrees at a speed of 1 degree/minute; the phi-axis was spinning at an angle of 360 degrees at a speed of 2 degrees/second; 0.3 mm collimator; the collection time was 60 minutes; the temperature was room temperature; and the heater was not used. The sample was presented to the X-ray source in a boron rich glass capillary.

In addition, the analysis parameters were as follows: the integration 2-theta range was 2-60 degrees; the integration chi range was 0-360 degrees; the number of chi segments was 1; the step size used was 0.02; the integration utility was cylint; normalization was used; dark counts were 8; omega offset was 180; and chi and phi offsets were 0.

PXRD diffractograms were also acquired via the Bruker AXS D8 Discover X-ray Diffractometer. This instrument was equipped with GADDS™ (General Area Diffraction Detection System), a Bruker AXS HI-STAR Area Detector at a distance of 15.05 cm as per system calibration, a copper source (Cu/K_{α} 1.54056 angstroms), automated x-y-z stage, and 0.5 mm collimator. The sample was compacted into pellet form and mounted on the x-y-z stage. A diffractogram was acquired under ambient condition a at a powder setting of 40 kV and 40 mA in reflection mode while the sample remained stationary. The exposure time was varied and specified for each sample. The diffractogram obtained underwent a spatial remapping procedure to account for the geometrical pincushion distortion of the area detector then integrated along chi from −118.8 to −61.8 degrees and 2-theta 2.1-37 degrees at a step size of 0.02 degrees with normalization set to bin normalize.

The relative intensity of peaks in a diffractogram is not necessarily a limitation of the PXRD pattern because peak intensity can vary from sample to sample, e.g., due to crystalline impurities. Further, the angles of each peak can vary by about +/−0.1 degrees, or about +/−0.05. The entire pattern or most of the pattern peaks may also shift by about +/−0.1 degree due to differences in calibration, settings, and other variations from instrument to instrument and from operator to operator.

Single crystal X-ray crystallographic analyses conducted in connection with the experiments described herein were used to determine unit cell dimensions, space group, and atomic position of all atoms in a compound relative to the origin of its unit cell. The unit cell dimension is defined by three parameters; length of the sides of the cell, relative angles of sides to each other and the volume of the cell. The lengths of the sides of the unit cell are defined by a, b and c. The relative angles of the cell sides are defined by alpha, beta, and gamma. The volume of the cell is defined as V. A more detailed account of unit cells can be found in Chapter 3 of Stout & Jensen, X-Ray Structure Determination; A Practical Guide, Mac Millian Co., New York, N.Y. (1968).

The results of a single crystal X-ray analysis are limited to the crystal placed in the X-ray beam. Crystallographic data on a large group of crystals provides powder X-ray diffraction. If the powder is a pure crystalline compound a simple powder diagram is obtained. To compare the results of a single crystal analysis and powder X-ray analysis a simple calculation can be done converting the single crystal data into a powder X-ray diagram, SHELXTL Plus® computer program, Reference Manual by Siemens Analytical X-ray Instrument, Chapter 10, p. 179-181, 1990. This conversion is possible because the single crystal experiment routinely determines the unit cell dimensions, space group, and atomic positions. These parameters provide a basis to calculate a perfect powder pattern. Comparing this calculated powder pattern and the powder pattern experimentally obtained from a large collection of crystals will confirm if the results of the two techniques are the same.

Single crystal x-ray data were collected on a Bruker SMART-APEX CCD diffractometer (M. J. Zaworotko, Department of Chemistry, University of South Florida). Lattice parameters were determined from least squares analysis. Reflection data was integrated using the program SAINT. The structure was solved by direct methods and refined by full matrix least squares using the program SHELXTL (Sheldrick, G. M. SHELXTL, Release 5.03; Siemans Analytical X-ray Instruments Inc.: Madison, Wis.).

For PXRD data herein, including Tables and Figures, each composition of the present invention may be characterized by any one, any two, any three, any four, any five, any six, any seven, or any eight or more of the 2 theta angle peaks. Any one, two, three, four, five, or six DSC transitions can also be used to characterize the compositions of the present invention. TGA data can also be used to characterize the compositions of the present invention. Likewise, single-crystal x-ray data can also be used to characterize the compositions of the present invention. Different combinations of the PXRD peaks and the DSC transitions can also be used to characterize the compositions. Any of the above analytical techniques can be combined with another technique to characterize a composition of the present invention.

### EXAMPLE 1

### Sagerconazole

### Comparative Data

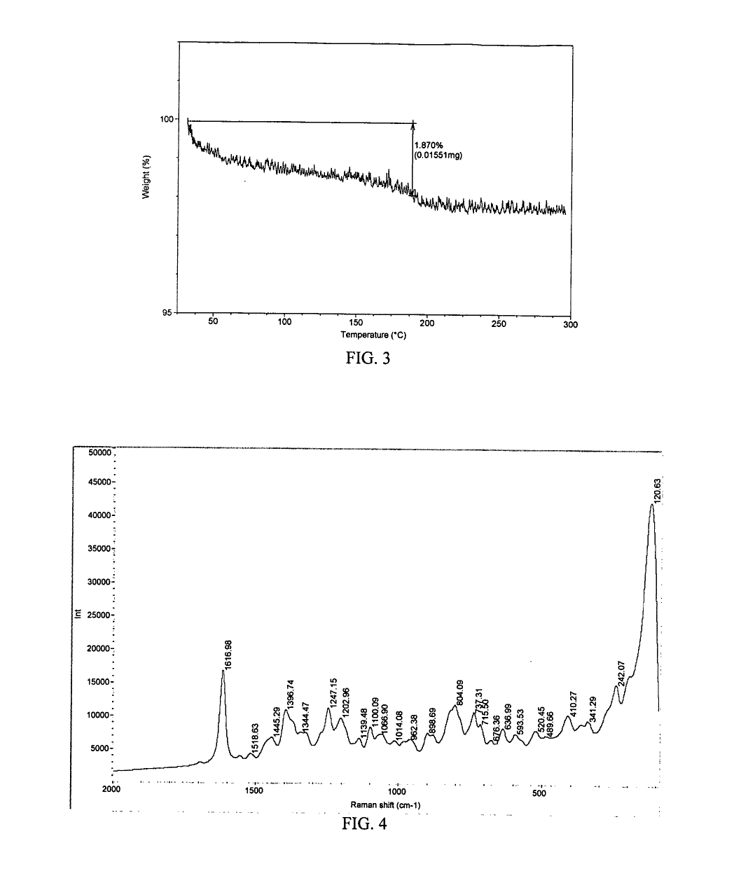

Saperconazole was analyzed via DSC, TGA, Raman, and PXRD. (The preparation of saperconazole is described in U.S. Pat. No. 4,916,134.) The DSC thermogram showed an endothermic transition at about 189 degrees C. (See FIG. 2). The TGA thermogram showed about a 1.9 percent weight loss between about 30 and about 183 degrees C. (See FIG. 3). Saperconazole can be characterized by any one, any two, any three, any four, any five, or any six or more of the peaks in the Raman spectrum in FIG. 4 including, but not limited to, the peaks at about 1617, 1397, 1247, 1203, 1100, 899, 804, 737, 716, 410, and 341 cm⁻¹. Saperconazole can be characterized by any one, any two, any three, any four, any five, or any six or more of the peaks in the PXRD diffractogram in FIG. 5 including, but not limited to, 3.05, 6.19, 8.21, 9.31, 10.95, 16.77, 17.75, 18.95, 19.27, 21.03, 21.85, 23.23, and 26.79 degrees 2-theta.

### EXAMPLE 2

### Saperconazole:DL-Tartaric Acid Co-Crystal

About 10 mg saperconazole and approximately 1 molar equivalent of DL-tartaric acid were dissolved in 200 microliters THF or 100 microliters 1,4-dioxane (dioxane) by heating at 75 degrees C. for approximately 2 hours. The solutions were cooled to 5 degrees C. and incubated until solid recrystallized. The solid was determined to be saperconazole:DL-tartaric acid co-crystal.

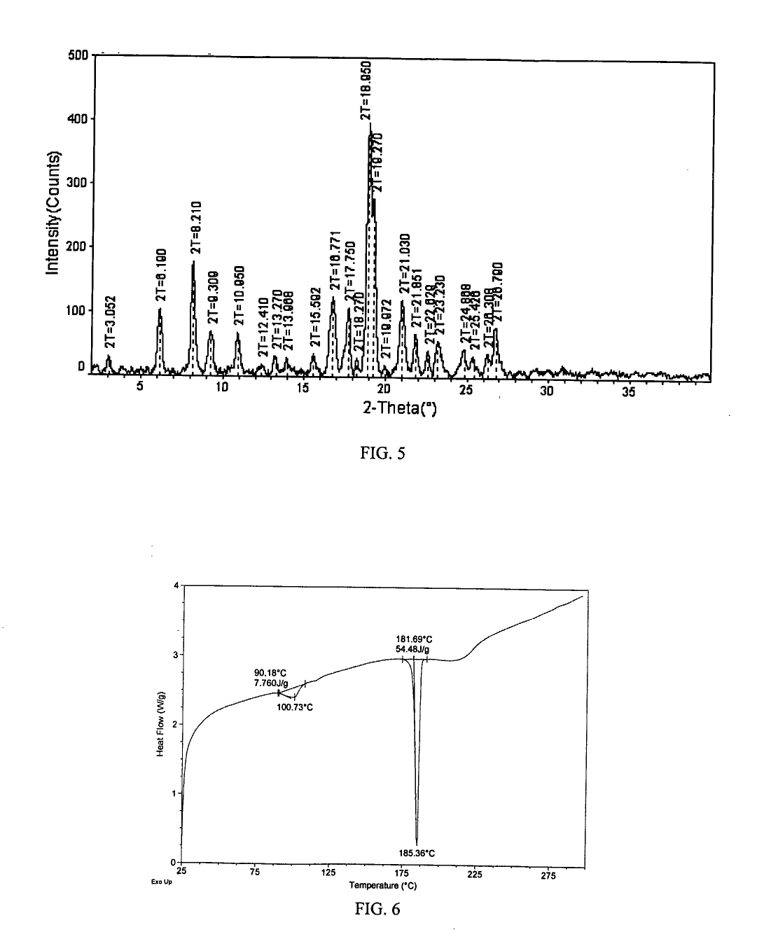

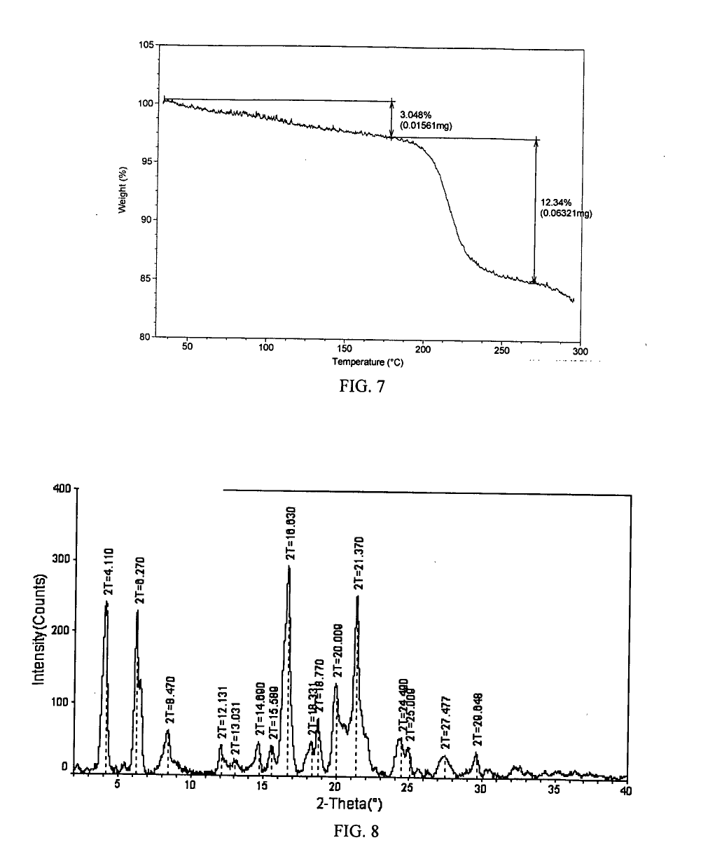

The saperconazole:DL-tartaric acid co-crystal was analyzed via DSC, TGA, and PXRD. The DSC thermogram showed an endothermic transition at about 185 degrees C. (See FIG. 6). The TGA thermogram showed about a 12.3 percent weight loss between about 175 and about 270 degrees C. (See FIG. 7). The saperconazole:DL-tartaric acid co-crystal can be characterized by any one, any two, any three, any four, any five, or any six or more of the peaks in the PXRD diffractogram in FIG. 8 including, but not limited to, 4.11, 6.27, 8.47, 14.69, 16.63, 18.33, 18.77, 20.01, 21.37, and 24.49 degrees 2-theta.

### EXAMPLE 3

### Saperconazole:Succinic Acid Co-Crystal

In a first synthesis, about 10 mg saperconazole and approximately 1 molar equivalent of succinic acid were dissolved in 200 microliters dioxane by heating at 75 degrees C. for approximately 2 hours. The solution was cooled to 5 degrees C. and incubated until solid crystallized and was then characterized. In a second synthesis, about 4 mg saperconazole and approximately 1 molar equivalent of succinic acid were dissolved in 100 microliters dioxane by heating the sample at approximately 70 degrees C., and immediately upon dissolution the solution was slowly cooled to 5 degrees C. The solution was incubated until very small needles crystallized. 100 microliters 1,2-dichloroethane were added, and the vial was held under warm running water to redissolve the solid. The solution was again incubated at 5 degrees C. until solid crystallized and was then characterized.

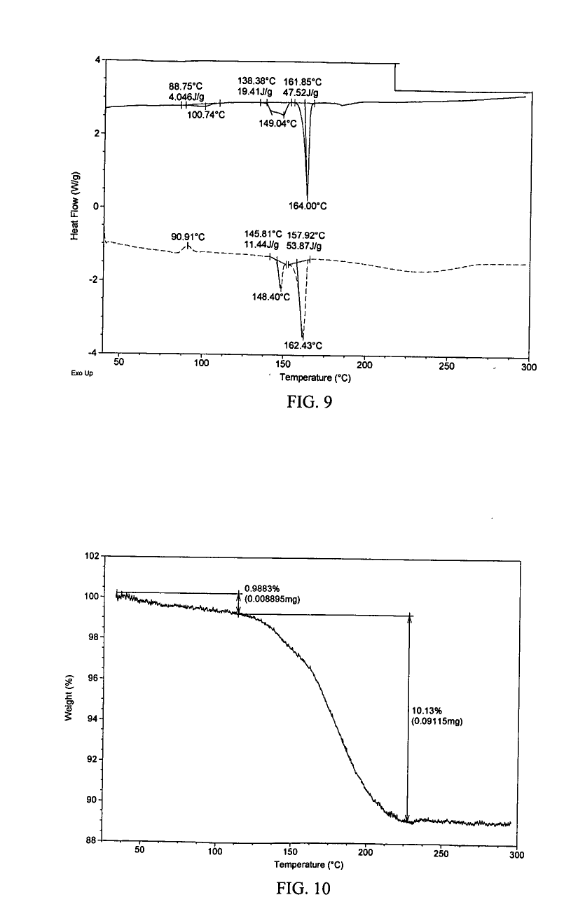

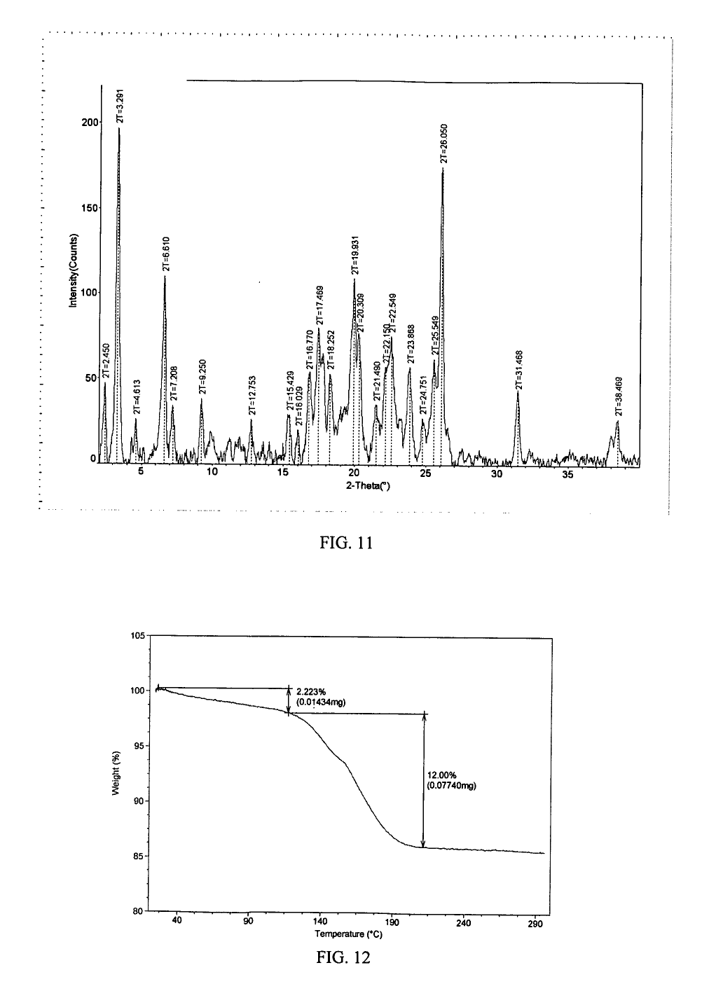

The resultant solid from the first synthesis, above, was analyzed via DSC, TGA, and PXRD. The DSC thermogram showed an endothermic transition at about 149 degrees C. and another endothermic transition at about 164 degrees C. (See FIG. 9, top trace). The TGA thermogram showed about a 10.1 percent weight loss between about 112 and about 225 degrees C. (See FIG. 10). The solid can be characterized by any one, any two, any three, any four, any five, or any six or more of the peaks in the PXRD diffractogram in FIG. 11 including, but not limited to, 2.45, 3.29, 6.61, 9.25, 15.43, 16.77, 17.47, 18.25, 19.93, 20.31, 22.55, 23.87, 26.05, and 31.47 degrees 2-theta.

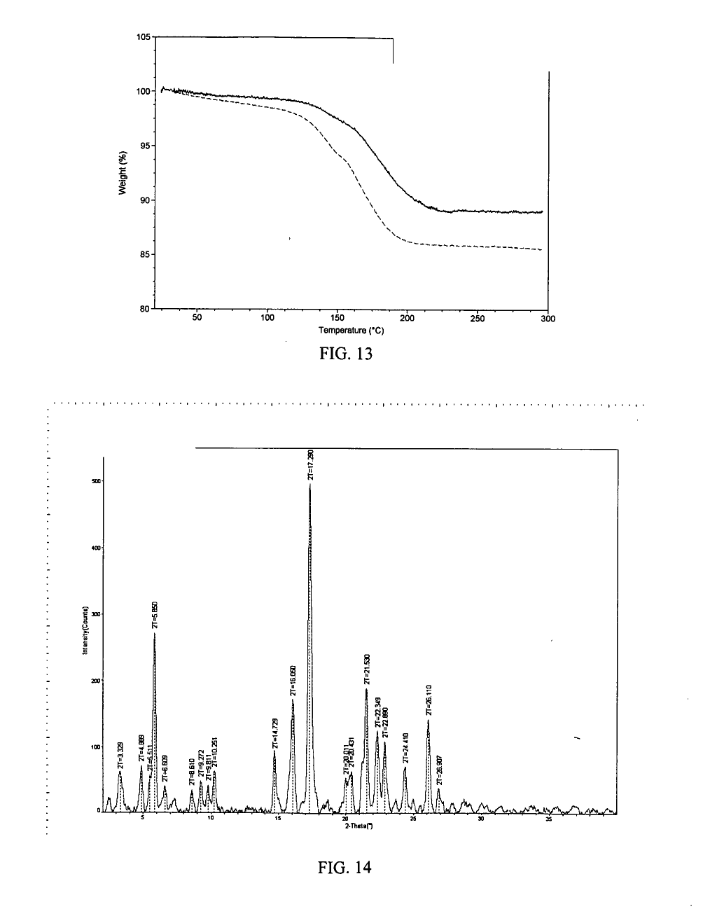

The resultant solid from the second synthesis, above, was analyzed via DSC, TGA, and PXRD. The DSC thermogram showed an endothermic transition at about 148 degrees C. and another endothermic transition at about 162 degrees C. (See FIG. 9, bottom trace). The TGA thermogram showed about a 12.0 percent weight loss between about 112 and about 225 degrees C. (See FIG. 12). The solid can be characterized by any one, any two, any three, any four, any five, or any six or more of the peaks in the PXRD diffractogram in FIG. 14 including, but not limited to, 3.33, 4.89, 5.85, 8.61, 9.27, 9.81, 10.25, 14.73, 16.05, 17.29, 20.43, 21.53, 22.35, 22.89, 24.41, and 26.11 degrees 2-theta. FIG. 13 shows the TGA thermograms of both solids, top trace corresponds to the resultant solid of the first synthesis, bottom trace corresponds to the resultant solid of the second synthesis.

Based on these data, the resultant solids from each of the above methods may be two distinct forms of the saperconazole:succinic acid co-crystal. For example, the PXRD diffractograms of each solid appear to display several important differences. In order to further elucidate the characteristics of the saperconazole:succinic acid co-crystal, additional PXRD data were taken.

Additional PXRD data were used to construct the unit cell for the saperconazole:succinic acid co-crystal. The resultant solid from the first synthesis, above, was allowed to sit for about 6 weeks. After that time, a second PXRD diffractogram was obtained. This second PXRD was the same as that obtained from the second synthesis, above. Based on this diffractogram, a triclinic unit cell with a=21.2 angstroms, b=18.3 angstroms, c=9.9 angstroms, alpha=71.9 degrees, beta=101.4 degrees, gamma=121.4 degrees, and a volume of about 3119 cubic angstroms was determined for the saperconazole:succinic acid co-crystal.

### EXAMPLE 4

### Saperconazole:L-Malic Acid Co-Crystal

About 10 mg saperconazole and approximately 1 molar equivalent of L-malic acid were dissolved in 200 microliters dioxane by heating at 75 degrees C. for approximately 2 hours. The solution was cooled to 5 degrees C. and incubated until solid crystallized and was then characterized. The solid was determined to be saperconazole:L-malic acid co-crystal.

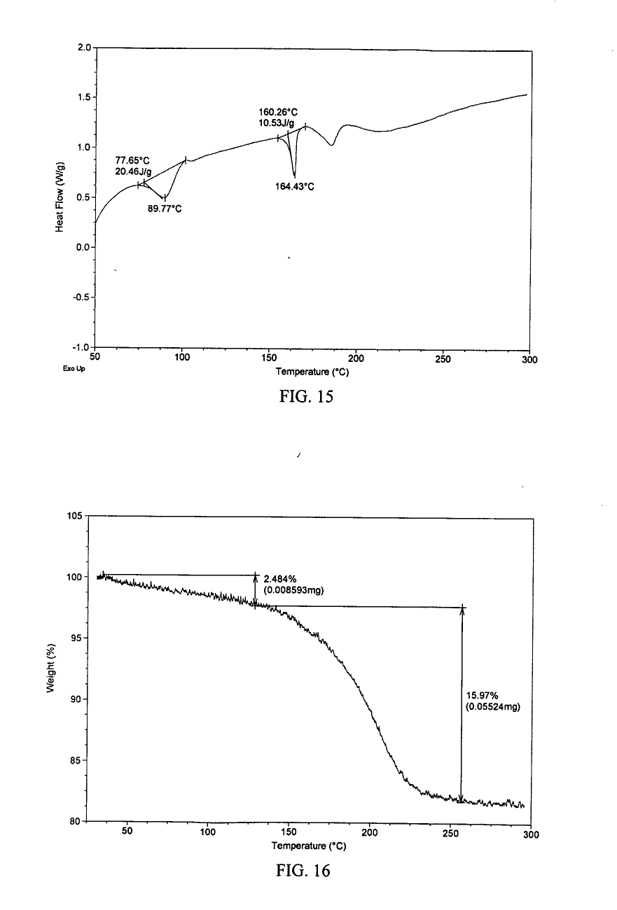

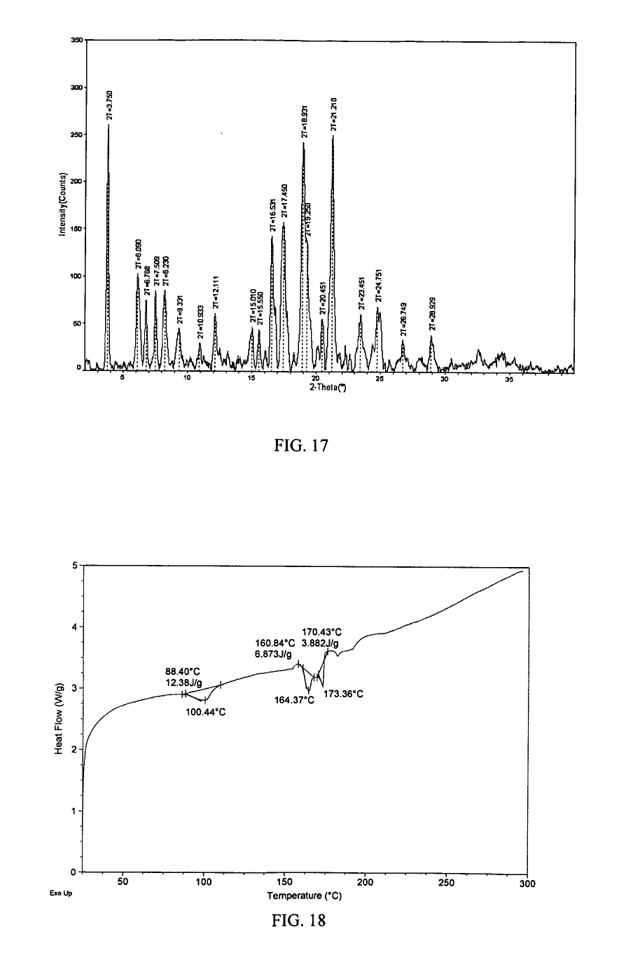

The saperconazole:L-malic acid co-crystal was analyzed via DSC, TGA, and PXRD. The DSC thermogram showed an endothermic transition at about 164 degrees C. (See FIG. 15). The TGA thermogram showed about a 16.0 percent weight loss between about 130 and about 255 degrees C. (See FIG. 16). The saperconazole:L-malic acid co-crystal can be characterized by any one, any two, any three, any four, any five, or any six or more of the peaks in the PXRD diffractogram in FIG. 17 including, but not limited to, 3.75, 6.09, 6.77, 7.51, 8.23, 9.33, 12.11, 16.53, 17.45, 18.93, 21.21, 23.45, and 24.75 degrees 2-theta.

### EXAMPLE 5

### Saperconazole:Fumaric Acid Co-Crystal

About 10 mg saperconazole and approximately 1 molar equivalent of fumaric acid were dissolved in 200 microliters dioxane by heating at 75 degrees C. for approximately 2 hours. The solution was cooled to 5 degrees C. and incubated until solid crystallized and was then characterized. The solid was determined to be saperconazole:fumaric acid co-crystal.

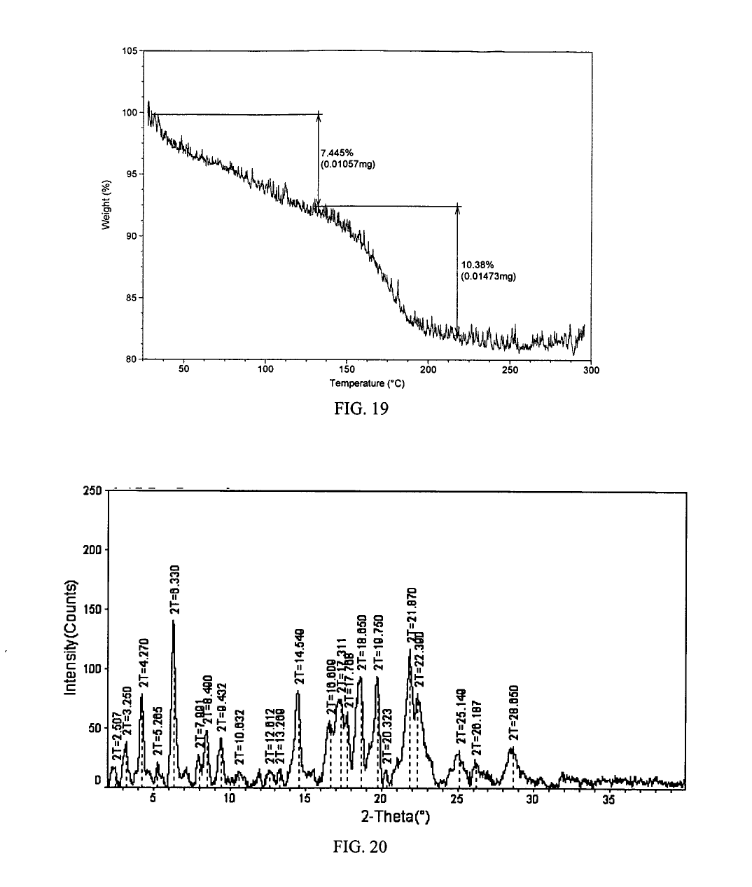

The saperconazole:fumaric acid co-crystal was analyzed via DSC, TGA, and PXRD. The DSC thermogram showed an endothermic transition at about 164 degrees C. and another endothermic transition at about 173 degrees C. (See FIG. 18). The TGA thermogram showed about a 10.4 percent weight loss between about 130 and about 220 degrees C. (See FIG. 19). The saperconazole:fumaric acid co-crystal can be characterized by any one, any two, any three, any four, any five, or any six or more of the peaks in the PXRD diffractogram in FIG. 20 including, but not limited to, 2.51, 3.25, 4.27, 6.33, 8.49, 9.43, 14.55, 16.61, 17.31, 17.77, 18.65, 19.75, 21.87, and 22.39 degrees 2-theta.

### EXAMPLE 6

### Saperconazole:Glutaric Acid Co-Crystal

About 5 mg saperconazole and approximately 1 molar equivalent of glutaric acid were dissolved in 200 microliters dioxane by heating at 75 degrees C. for approximately 2 hours. The solutions were cooled to 5 degrees C. and incubated until solid recrystallized and was then characterized. The solid was determined to be saperconazole:glutaric acid co-crystal.

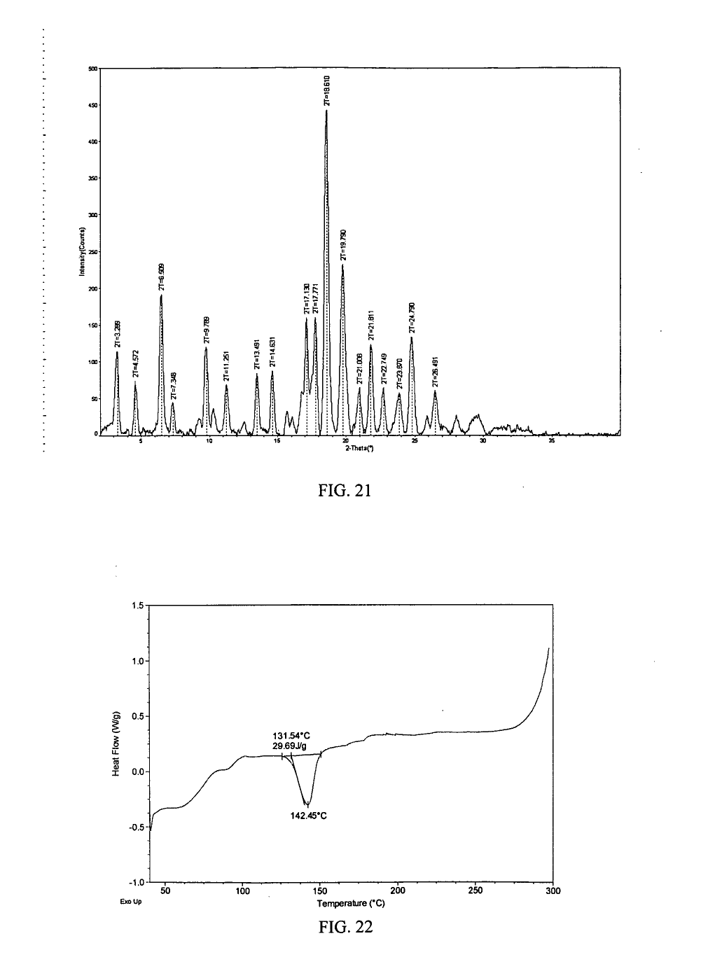

The saperconazole:glutaric acid co-crystal was analyzed via PXRD. The saperconazole:glutaric acid co-crystal can be characterized by any one, any two, any three, any four, any five, or any six or more of the peaks in the PXRD diffractogram in FIG. 21 including, but not limited to, 3.29, 4.57, 6.51, 9.79, 11.25, 13.49, 14.63, 17.13, 17.77, 18.61, 19.79, 21.01, 21.81, 22.75, 23.87, 24.79, and 26.49 degrees 2-theta.

### EXAMPLE 7

### Saperconazole Mesylate Salt

About 10 mg saperconazole and approximately 1 molar equivalent methanesulfonic acid were dissolved in 200 microliters ethanol by heating at 75 degrees C. for approximately 2 hours. The solution was cooled to 5 degrees C. and incubated until solid recrystallized and was then characterized. The solid was determined to be saperconazole mesylate.

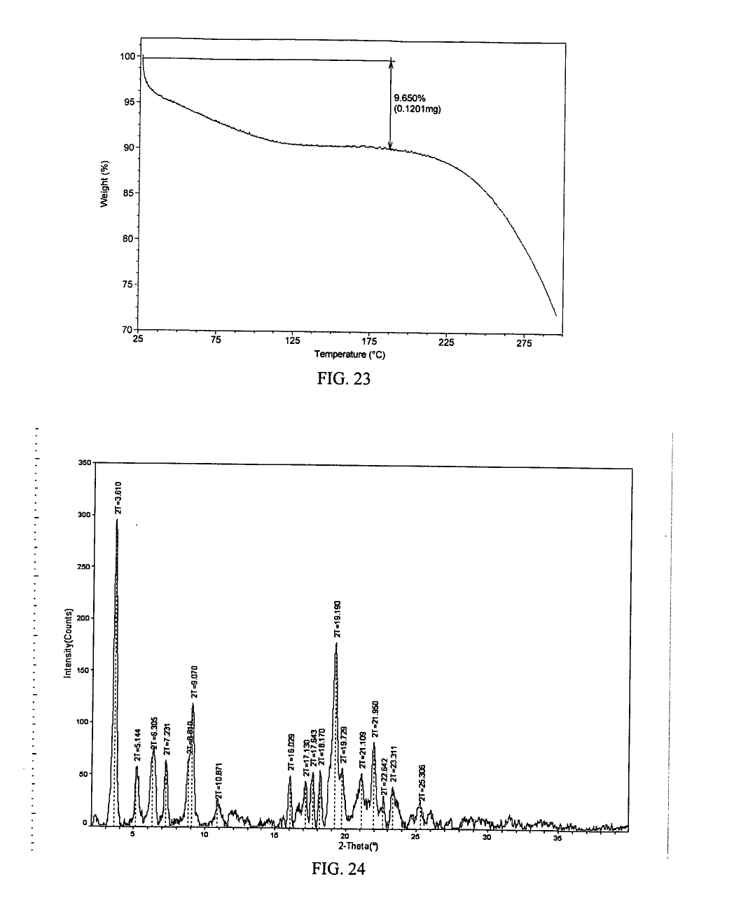

The saperconazole mesylate salt was analyzed via DSC, TGA, and PXRD. The DSC thermogram showed an endothermic transition at about 142 degrees C. (See FIG. 22). The TGA thermogram showed about a 9.7 percent weight loss between about room temperature and about 190 degrees C. (See FIG. 23). The saperconazole mesylate salt can be characterized by any one, any two, any three, any four, any five, or any six or more of the peaks in the PXRD diffractogram in FIG. 24 including, but not limited to, 3.61, 5.14, 6.31, 7.23, 9.07, 16.03, 17.13, 17.64, 18.17, 19.19, 21.11, 21.95, and 23.31 degrees 2-theta.

1-18. (canceled)

19. A co-crystal comprising saperconazole and a carboxylic acid.

20. The co-crystal of claim 19, wherein said carboxylic acid is tartaric acid.

21. The co-crystal of claim 20, wherein said co-crystal exhibits a powder X-ray diffractogram comprising peaks at about 4.11 and 6.27 degrees 2-theta.

22. The co-crystal of claim 20, wherein said co-crystal exhibits a powder X-ray diffractogram comprising peaks at about 6.27, 8.47, 16.63, and 20.01 degrees 2-theta.

23. The co-crystal of claim 20, wherein said co-crystal exhibits a powder X-ray diffractogram comprising peaks at about 4.11, 6.27, 8.47, 16.63, 20.01, and 21.37 degrees 2-theta.

24. The co-crystal of claim 19, wherein said carboxylic acid is succinic acid.

25. The co-crystal of claim 24, wherein said co-crystal exhibits a powder X-ray diffractogram comprising peaks at about 3.33 and 5.85 degrees 2-theta.

26. The co-crystal of claim 24, wherein said co-crystal exhibits a powder X-ray diffractogram comprising peaks at about 4.89, 5.85, 14.73, and 21.53 degrees 2-theta.

27. The co-crystal of claim 24, wherein said co-crystal exhibits a powder X-ray diffractogram comprising peaks at about 5.85, 10.25, 14.73, 16.05, 17.29, and 21.53 degrees 2-theta.

28. The co-crystal of claim 24, wherein said co-crystal exhibits a powder X-ray diffractogram comprising peaks at about 3.29, 6.61, 9.25, 17.47, 19.93, and 26.05 degrees 2-theta.

29. The co-crystal of claim 19, wherein said carboxylic acid is malic acid.

30. The co-crystal of claim 29, wherein said co-crystal exhibits a powder X-ray diffractogram comprising peaks at about 3.75 and 6.09 degrees 2-theta.

31. The co-crystal of claim 29, wherein said co-crystal exhibits a powder X-ray diffractogram comprising peaks at about 3.75, 6.77, 8.23, and 16.53 degrees 2-theta.

32. The co-crystal of claim 29, wherein said co-crystal exhibits a powder X-ray diffractogram comprising peaks at about 3.75, 6.09, 7.51, 12.11, 16.53, and 17.45 degrees 2-theta.

33. The co-crystal of claim 19, wherein said carboxylic acid is fumaric acid.

34. The co-crystal of claim 33, wherein said co-crystal exhibits a powder X-ray diffractogram comprising peaks at about 4.27 and 6.33 degrees 2-theta.

35. The co-crystal of claim 33, wherein said co-crystal exhibits a powder X-ray diffractogram comprising peaks at about 6.33, 9.43, 17.31, and 19.75 degrees 2-theta.

36. The co-crystal of claim 33, wherein said co-crystal exhibits a powder X-ray diffractogram comprising peaks at about 4.27, 6.33, 9.43, 14.55, 18.65, and 19.75 degrees 2-theta.

37. The co-crystal of claim 19, wherein said carboxylic acid is glutaric acid.

38. The co-crystal of claim 37, wherein said co-crystal exhibits a powder X-ray diffractogram comprising peaks at about 3.29 and 4.57 degrees 2-theta.

39. The co-crystal of claim 37, wherein said co-crystal exhibits a powder X-ray diffractogram comprising peaks at about 4.57, 11.25, 13.49, and 18.61 degrees 2-theta.

40. The co-crystal of claim 37, wherein said co-crystal exhibits a powder X-ray diffractogram comprising peaks at about 3.29, 6.51, 9.79, 13.49, 14.63, and 19.79 degrees 2-theta.
